(19) United States
(12) Patent Application Publication
(10) Pub. No.: US 2020/0126640 A1
(43) Pub. Date: Apr. 23, 2020

(54) COMPUTATIONAL AFFINITY MATURATION OF ANTIBODY LIBRARIES USING SEQUENCE ENUMERATION

(71) Applicant: IGC BIO, INC., Cambridge, MA (US)

(72) Inventor: Lior ZIMMERMAN, Tel Aviv (IL)

(73) Assignee: IGC BIO, INC., Cambridge, MA (US)

(21) Appl. No.: 16/656,113

(22) Filed: Oct. 17, 2019

Related U.S. Application Data
(60) Provisional application No. 62/746,985, filed on Oct. 17, 2018.

(51) Int. Cl. G16C 20/50; G16C 60/00; G16C 20/20; G16C 20/70; C07K 16/06; C12N 15/10; C40B 30/04

(52) U.S. Cl. CPC G16C 20/50; G16C 60/00; G16C 20/20; G16C 20/70; C07K 16/065; C07K 2317/32; C12N 15/1058; C40B 30/04; C07K 2317/92; C07K 2317/94; C07K 2317/565; C12N 15/1089

(57) ABSTRACT

Methods for computational affinity maturation are described wherein a candidate antibody sequence is optimized for affinity, stability, or both, first utilizing computational saturation mutagenesis to identify and mutations at those positions that satisfy a predefined first threshold of affinity score or stability score, generating sequences comprising all combinations of the mutations at each position, computing the affinity score, stability score, or both and ranking the variant antibody sequences according to a predefined second threshold, then from the affinity score or stability score of all generated sequences identify the optimized antibody sequences. The method is ideally carried out on multiple CPUs where a server generates the sequences, the CPUs evaluate the affinity or stability score, reports the results to the server, which ranks all of the data from the CPUs to identify the optimal candidates. Optimal candidates can then be expressed and experimentally evaluated to identify future clinical or research reagent antibodies.

### CROSS-REFERENCE TO RELATED APPLICATIONS

This application claims the benefit of U.S. Provisional Patent Application No. 62/746,985, filed Oct. 17, 2018, which is incorporated herein by reference in its entirety.

### BACKGROUND OF THE INVENTION

Antibodies are the most versatile class of binding molecule known and have numerous applications in biomedicine. Due to their versatility, dozens of antibodies are in routine clinical use to diagnose and treat the most intransigent diseases and thousands more are used as research reagents. These antibodies were all isolated either by animal immunization or from synthetic repertoires that mimic the diversity of vertebrate immune systems. Notwithstanding these successes, however, natural repertoires have limitations, including biases and redundancy in representing the vast potential sequence and conformation space available to antibodies, and many antibodies exhibit polyspecificity and low expressibility, failing to meet the stringent requirements of research or clinical use (Xu Y., et al. Addressing polyspecificity of antibodies selected from an in vitro yeast presentation system: A FACS-based, high-throughput selection and analytical tool. Protein Eng Des Sel. 2013; 26:663-670; Bradbury A, Plückthun A. Reproducibility: Standardize antibodies used in research. Nature. 2015; 518:27-29; Jain T, et al. Biophysical properties of the clinical-stage antibody landscape. Proc Natl Acad Sci USA, 2017; 114:944-949). In addition, targeting the correct epitope is a critical step in selection of a monoclonal antibody to achieve the desired mechanism of action.

In order to find a candidate that fulfills all the biophysical and biochemical requirements to work in clinical/research settings, each potential candidate antibody must go through extensive screenings and biophysical optimizations (Hoogenboom, H. R. Selecting and screening recombinant antibody libraries. Nat. Biotechnol. 23, 1105-1116 (2005); Carter, P. J. Potent antibody therapeutics by design. Nat. Rev. Immunol. 6, 343-357 (2006)). The poor efficiency and speed of this process rarely generates candidates that become approved therapeutics or diagnostics, let alone ubiquitous research reagents.

It has been, therefore, a long-standing goal to develop a method that has the capability to optimize all the requirements of affinity, specificity, stability, epitope and binding mode simultaneously a priori, shortening the time-consuming optimization steps. Recently, Baran et al described a computational platform for de-novo epitope specific antibody design (Baran D, Pszolla M G, Lapidoth G D, et al. Principles for computational design of binding antibodies. Proceedings of the National Academy of Sciences of the United States of America, 2017; 114(41):10900-10905. doi: 10.1073/pnas.1707171114). The platform works by using first principles, (i.e. biophysical modeling of protein molecules) to design novel antibody structures to bind predefined epitopes. This method, however, produced low affinity antibodies that required further affinity maturation, although a handful of point mutations that were introduced by error-prone PCR were fortuitous in lowering the Kd to the nM range.

It is toward improving the efficiency of computational-bases antibody design including the ability to capture long range electrostatics and backbone movements that are induced by the binding event, and computationally parallel processing a vast number of sequences concurrently to maximize the identification of improved variant antibody sequences, that the present invention is directed.

### BRIEF DESCRIPTION OF THE INVENTION

The invention is directed to a computer implemented method for generating a library of variant, epitope-specific antibody sequences that have a predicted improved affinity, stability, or the combination thereof over the original epitope-specific antibody sequence, the method comprising the steps of:
a. utilizing computational saturation mutagenesis, identify positions in said original antibody sequence, and mutations at said positions of said original epitope-specific antibody sequence, that satisfy a predefined first threshold of affinity score, stability score, or combination thereof compared to that of the original epitope-specific antibody sequence;
b. for each of said positions and mutations at said positions, compute the affinity score, stability score, or the combination thereof, of a plurality of variant antibody sequences having each mutation at each of said positions, and rank the variant antibody sequences having each of said mutations at each of said positions according to a predefined second threshold of affinity score, stability score, or combination thereof compared to that of the original antibody sequence; and
c. identify from an affinity score, stability score, or combination thereof that is above a predefined third threshold of affinity score, stability score or combination thereof compared to that of the original antibody sequence those variant antibody sequences with a predicted improved affinity score, stability score or combination thereof compared to that of the original antibody sequence.

In one embodiment, the original epitope-specific antibody sequence is obtained from a computational model of the antibody-antigen complex. In one embodiment, the model is obtained from use of methods described in PCT/US18/12721, wherein based on a predetermined epitope, one or more seed structures are generated based on one or more predetermined amino acid sequences of a complementarity determining region (CDR), one or more predetermined variable heavy (VH) and variable light (VL) structural framework (VHN L) pairs, or a combination thereof. The one or more seed structures is docked on the predetermined epitope, followed by evaluating one or more motifs of said one or more seed structures for one or more predetermined developability properties; and identifying one or more target structures to generate a library of antibodies structures based on the preselected epitope. The computational antibody-antigen model developed during this method is then used to identify positions in the antibody that are candidates for improvement by mutation, and areas les amenable to such modifications.

In one embodiment, in step (a), said positions that are identified by computational saturation mutagenesis are determined using a position specific scoring matrix (PSSM), wherein one or more replacement amino acids at each position results in a negative, unchanged, or slightly positive affinity score or stability score compared to the amino acid at that position in the original epitope-specific antibody sequence.

In one embodiment, the positions identified in the original epitope-specific antibody sequence are within about 8 A of the antigen.

In one embodiment, in step (b), computation of the affinity score or stability score of the plurality of variant epitope-specific antibody sequences is carried out on multiple computers, such that each computer computes, for a portion of the plurality of variant antibody sequences, the difference in affinity score or stability score of said subset of variant antibody sequences and that of the original epitope-specific antibody sequence. In one embodiment, the affinity score or stability score is determined using Rosetta protein modeling software.

In one embodiment, the first threshold of affinity or stability is a predefined value of the difference between the score before mutation and the score after mutation. In one embodiment, the second threshold for affinity or stability is a predefined value of difference in affinity or stability score before mutation and the affinity score or stability score after mutation. In one embodiment, the third threshold for affinity or stability is a predefined value of difference in affinity score or stability score before mutation and the affinity score or stability score after mutation.

In any of the embodiments described herein, the threshold is based on both the affinity score and the stability score.

In one embodiment, in a subsequent step, display methods such as yeast or phage display are used to experimentally evaluate the affinity of the antibody sequences to the antigen. In one embodiment, a fourth threshold may be used to identify candidate antibody sequences with an improved property for further evaluation or development for clinical or research use.

In one embodiment, the first threshold allows for selection of variant sequences that will be further evaluated for improved affinity, stability or the combination of both. In one embodiment, the second threshold is selected to identify candidate sequences with computationally identified improved affinity, stability or the combination of both, and said candidate sequences combined with other candidate sequences meeting the same threshold. In one embodiment, the third threshold is selected to identify the sequences having the most improved computationally identified affinity, stability of combination of both, for output from the method. In one embodiment, the fourth threshold is selected to identify candidate sequences having the experimentally determined improved affinity, stability or the combination of both. A candidate sequence above the fourth threshold may be a candidate for development for a clinical or research use.

In one embodiment, a method is provided for identifying an improved epitope-specific variant antibody sequence comprising the steps of:
a. obtaining a library of variant, epitope-specific antibody sequences that have a predicted improved affinity or stability over the original epitope-specific antibody sequence in accordance with the teachings described herein above;
b. generate DNA oligonucleotide sequences comprising the CDR3 sequences of the sequences in said library, suitable for expression in an organism in which binding activity in a display or expression system is to be assessed;
d. express the corresponding library of scFVs comprising said CDR3; and
e. screen said display or expression system for binding of the epitope and identify therefrom one or more CDR3 comprising an improved epitope-specific variant antibody sequence.

In one embodiment, a threshold value for improvement is provided such that a candidate sequence is selected as improved if meeting or exceeding the threshold value.

In one embodiment, the original epitope-specific antibody is obtained by epitope-specific antibody engineering.

In one embodiment, the epitope-specific antibody engineering of any of the foregoing embodiments is carried out by:
a. generating one or more seed structures based on one or more predetermined amino acid sequences of a complementarity determining region (CDR), one or more predetermined variable heavy (VH) and variable light (VL) structural framework (VHN L) pairs, or a combination thereof;
b. providing a predetermined epitope;
c. docking said one or more seed structures on said epitope;
d. evaluating one or more motifs of said one or more seed structures for one or more predetermined developability properties; and
e. identifying one or more target structures in order to generate a library, thereby generating a library of antibodies.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1A-B depicts the algorithm carried out by the worker and the server;

FIG. 2 depicts the scalable architecture of the algorithm;

FIG. 3A-D describe an epitope specific library targeting hPD-1;

FIGS. 4A-C describe another epitope specific library targeting hPD-1;

FIGS. 5A-G describe an epitope specific library targeting VEGF;

FIGS. 6A-B describe an epitope specific library targeting hPD-1;

FIGS. 7A-C describe a chimeric, epitope specific library targeting PD-1;

FIGS. 8A-K describe an error-prone, epitope specific library targeting PD-1;

FIGS. 9A-H describe information on general libraries of antibodies against VEGF and TNFa; and

FIG. 10 summarizes the data from these studies.

### DETAILED DESCRIPTION OF THE INVENTION

This disclosure describes an in-silica affinity maturation protocol that capable of scaling to an arbitrary number of concurrent processes to explore vast number of combination of potentially favorable mutations. It receives as input a computational model of an antibody-antigen complex, computes positions in the antibody sequence suitable for mutations that increase binding affinity with minimal to no compromise of the biophysical stability of the protein to large extent, and enumerates sequences with combinations of such mutations. Subsequent computational evaluation of sequences with every combination of mutations for improved properties results in identifying sequences that fulfill a predefined threshold. Output sequences are candidates for experimental evaluation and selection of one or more variant antibody sequences for potential clinical or research use.

Computational affinity maturation achieves in silico the fine tuning of antibody design to achieve the desired purposes. Input from an antibody-antigen model permits the exploration of long-range electrostatics and backbone movement that are induced by the binding event, permitting optimization to nanomolar binding affinity. The vast number of combinations of potentially favorable mutations are evaluated by use of a massively hypermultiplexed processor, or as is currently more practical, having a server parsing the models to worker processors or computers to conduct the evaluation of affinity, stability or other desired improved property of the candidate sequence, then report the result back to a server that will identify from the worker output the best computationally designed candidates. Subsequently, among these the experimentally identified best candidate(s) can be identified by a display methodology in which the actual affinity, stability or other feature(s) of the antibody can be determined. The in silico steps of the invention are carried out following an algorithm.

The candidate antibody for enhancing a property may be obtained from an antigen-antibody model such that the interactions between the antigen/epitope and regions on the antibody, including the CDR and backbone regions, can be identified including long-range electrostatic interactions and effects of antigen binding on the backbone structure, can be calculated to identify mutations at certain positions in the CDR3 sequence that can potentially enhance the antibody properties. Even in using such an antibody-antigen structure/space model to limit the number of potential mutations, the number of candidate sequences is still enormous and would require massive computing power to complete the task in a reasonable time frame.

Since the number of sequences that are possible, even if allowing just a handful of positions to be subjected to mutation, is enormous (10 mutable positions, each with 10-8 allowed amino acids, can easily reach 10⁸ different scfv sequences) a parallel computation strategy is adopted that is able to run on an arbitrary number of CPUs, enabling x1000-x5000 decrease in computation time.

If, for example, 10 positions in the antibody are each a candidate for 10 variant amino acids, the number of variants to be tested is 10\hat{ }x. Of course, at each mutable position of the sequence a different number of variants may be identified; the actual number can be calculated once the PSSM matrix is created and scores generated, and the total number of variants needing evaluation by the workers identified; based on the number of workers the time required to evaluate all variants is determined and the server can parse these in measured sets to the workers to conduct the evaluation in parallel.

Sets of potential improved antibody sequences generated by the server are then distributed to individual workers for evaluation. The worker, using the PDB structure of the antigen-antibody complex, the list of mutations at each position outputted from computational saturation mutagenesis a described above) is evaluated for the difference in affinity or stability between the sequence before mutation and the sequence after mutation. The mutations and their scores are returned to the server. The threshold value for an improved sequence would be more stringent than the PSSM score for identifying mutations; a negative value represents an improved score, and a threshold of −1, for example, will identify the most favorable variants.

After computation design is completed, sequences can be screened experimentally for affinity for the epitope and/or other properties, and the optimal sequences selected as leads for clinical or research reagent development.

Each of the steps in the method are described below.

Positions suitable for mutation can be identified from an antibody-antigen computation model such as that provided by the epitope-specific antibody engineering method described in PCT/US2018/12721, wherein using a predetermined epitope, one or more seed structures are generated based on one or more predetermined amino acid sequences of a complementarity determining region (CDR), one or more predetermined variable heavy (VH) and variable light (VL) structural framework (VHN L) pairs, or a combination thereof; docking the one or more seed structures on the epitope; and evaluating one or more motifs of the one or more seed structures for one or more predetermined properties such as affinity, stability, expression rate, immunogenicity and serum half-life. From the evaluation, one or more target structures can be identified, and an antibody library generated. The model of the antibody-antigen complex used in the development of the library is then used in the present invention for identifying sites in the antibody sequence that are candidates for improvement by mutation.

In one embodiment, a first amino acid sequence of a CDR associated with a heavy chain and a second amino acid sequence of a CDR associated with a light chain is obtained from a database of CDR sequences. In one embodiment, the first amino acid sequence is H3 sequence of CDR3. In another embodiment, the first amino acid sequence is L3 sequence of CDR3. One or more variable heavy (VH) and variable light (VL) structural framework (VHN L) pairs may be obtained. Each of the pair may have one or more predetermined developability properties that facilitate for screening antibodies. The predetermined developability properties may also facilitate for selecting one or more desirable VHN L pairs. Examples of a predetermined developability property include, for example, but not limited to, an expression rate (mg/L), a relative display rate, a thermal stability (Tm), an aggregation propensity, a serum half-life, an immunogenicity, and a viscosity. In a particular embodiment, the predetermined developability property is an immunogenicity. An analysis unit may facilitate analyzing the amino acid sequences and the VHNL pairs with the use of a macro-molecular algorithmic unit to generate one or more seed structures.

In one embodiment, the amino acid sequence of H3 loop, L3 loop, or a combination thereof can be modified or optimized based on a Point Specific Scoring Matrix (PSSM). In another embodiment, the amino acid sequence of H3 loop, L3 loop, or a combination thereof can be modified or optimized based on one or more VH/VL pairs. In one aspect, one or more seed structures are generated based on an energy function of H3 loop, L3 loop, VHNL pair or a combination thereof. In another aspect, one or more seed structures are generated based on humanization of the structures. A predetermined epitope may be provided. In one example, the epitope is determined based on a subset of a protein. In another example, the epitope has one or more residues that interact with its interacting partner at a predetermined distance. In one embodiment, the distance is <4A. Other suitable distances are also encompassed within the scope of the invention.

One or more seed structures may be docked on the epitope. Evaluation of the docked seed structures may be carried out for a shape complementarity and an epitope overlap. One or more seed structures having a value exceeding a predetermined threshold level may be selected. In one embodiment, the predetermined threshold level is based on a shape complementarity score. In another embodiment, the predetermined threshold level is based on an epitope overlap score. In some embodiments, the predetermined threshold level is based a combination of a shape complementarity score and an epitope overlap score.

In some embodiments, one or more selected seed structures can be optimized using a simulated annealing process which is an adaptation of the Monte Carlo method to generate sample states of a thermodynamic system. In another embodiment, the simulated annealing process is composed of rigid body minimization, antibody H3-L3 sequence optimization, optimizing the packing of interface and core, optimizing the backbone of antibody, optimizing the light and heavy chain orientation, optimizing the antibody as monomer, or a combination thereof.

Motif evaluation may facilitate evaluating one or more motifs of the selected structures to determine whether one or more motifs exhibit a negative effect for one or more predetermined developability properties. In some embodiments, the one or more motifs with negative effects are removed. In a particular embodiment, an immunogenic motif is removed.

In one embodiment, CDR regions are mutated according to a Point Specific Scoring Matrix (PSSM) and the evaluation may be performed by evaluating an energy score that is derived from the algorithmic unit. Library generation may facilitate identifying one or more target structures based on the determination of any negative effect of one or more motifs in order to generate a library. In one embodiment, the model used to provide the structure of the antibody-antigen complex is thus provided in order to carry out the subsequent steps in the method.

A computational saturation algorithm such as that described by Whitehead et al., Nature Biotechnology 2012; 30 (May):543-548, can be used to generate a list of amino acid candidates that can be introduced to positions deemed substitutable in the original antibody sequence. The input to the algorithm is an antibody structure (PDB file), a list of mutable positions, for each—a list of allowed amino acids, and optionally, a PSSM (Position Specific Scoring Matrix) that determines the likelihood of mutating position i to amino acid X (for each position i, for each amino acid X).

In the Whitehead et al. procedure, the calculated binding or folding energy for each single mutation was used to eliminate unfavorable mutations in the absence of any other mutations, generating a reduced subset of mutations for proceeding into further evaluation of combinations. In the present method, no such absolute elimination of unfavorable single mutations is performed in order to allow for combinations of mutations to potentially provide the desired improvements; instead, there is a tolerance for unfavorable mutations up to a predefined threshold. As a non-limiting example, in one embodiment, a benchmark is a threshold that equals one Rosetta energy unit (r.e.u.) using the “beta” energy function. Therefore, mutations are included in the combinatorial sequences if the estimated binding energy compared to the original sequence is favorable (i.e., negative), neutral, or minimally unfavorable (i.e., minimally positive), to allow combinations of such mutations to have a net positive effect on the desired property.

A “position-specific scoring matrix” (PSSM), also known in the art as position weight matrix (PWM), or a position-specific weight matrix (PSWM), is a commonly used representation of recurring patterns in biological sequences, based on the frequency of appearance of a character (monomer; amino acid; nucleic acid etc.) in a given position along the sequence. Thus, PSSM represents the log-likelihood of observing mutations to any of the 20 amino acids at each position. PSSMs are often derived from a set of aligned sequences that are thought to be structurally and functionally related and have become widely used in many software tools for computational motif discovery. In the context of amino acid sequences, a PSSM is a type of scoring matrix used in protein BLAST searches in which amino acid substitution scores are given separately for each position in a protein multiple sequence alignment. Thus, a Tyr-Trp substitution at position A of an alignment may receive a very different score than the same substitution at position B, subject to different levels of amino acid conservation at the two positions. This is in contrast to position-independent matrices such as the PAM and BLOSUM matrices, in which the Tyr-Trp substitution receives the same score no matter at what position it occurs. PSSM scores are generally shown as positive or negative integers. Positive scores indicate that the given amino acid substitution occurs more frequently in the alignment than expected by chance, while negative scores indicate that the substitution occurs less frequently than expected. Large positive scores often indicate critical functional residues, which may be active site residues or residues required for other intermolecular or intramolecular interactions. PSSMs can be created using Position-Specific Iterative Basic Local Alignment Search Tool (PSI-BLAST) [Schäffer, A. A. et al., Nucl. Acids Res., 2001, 29(14), pp. 2994-3005], which finds similar protein sequences to a query sequence, and then constructs a PSSM from the resulting alignment. Alternatively, PSSMs can be retrieved from the National Center for Biotechnology Information Conserved Domains Database (NCBI CDD) database, since each conserved domain is represented by a PSSM that encodes the observed substitutions in the seed alignments. These CD records can be found either by text searching in Entrez Conserved Domains or by using Reverse Position-Specific BLAST (RPS-BLAST), also known as CD-Search, to locate these domains on an input protein sequence.

In the context of some embodiments of the present invention, a PSSM data file can be in the form of a table of integers, each indicating how evolutionary conserved is any one of the 20 amino acids at any possible position in the sequence of the designed antibody. As indicated hereinabove, a positive integer indicates that an amino acid is more probable in the given position than it would have been in a random position in a random protein, and a negative integer indicates that an amino acid is less probable at the given position than it would have been in a random protein. In general, the PSSM scores are determined according to a combination of the information in the input MSA and general information about amino acid substitutions in nature, as introduced, for example, by the BLOSUM62 matrix [Eddy, S. R., Nat Biotechnol, 2004, 22(8):1035-6].

In general, the method presented herein can use the PSSM output of a PSI-BLAST software package to derive a PSSM for both the original MSA and all sub-MSA files. A final PSSM input file, according to some embodiments of the present invention, includes the relevant lines from each PSSM file. For sequence positions that represent a secondary structure, relevant lines are copied from the PSSM derived from the original full MSA. For each loop, relevant lines are copied from the PSSM derived from the sub-MSA file representing that loop. Thus, according to some embodiments of the present invention, a final PSSM input file is a quantitative representation of the sequence data, which is incorporated in the structural calculations, as discussed hereinbelow.

According to some embodiments of the present invention, MSA and PSSM-based rules determine the unsubstitutable positions and the substitutable positions in the amino acid sequence of the original polypeptide chain, and further determine which of the amino acid alternatives will serve as candidate alternatives in the single position scanning step of the method, as discussed hereinbelow.

The method, according to some embodiments of the present invention, allows the incorporation of information about the original antibody chain and/or the wild type antibody. This information, which can be provided by various sources including the computational model described above, is incorporated into the method as part of the rules by which amino acid substitutions are governed during the design procedure. Albeit optional, the addition of such information is advantageous as it reduces the probability of the method providing results which include folding- and/or function-abrogating substitutions.

To decrease the probability of sequences leading to misfolding during the sequence design process, residues that are known to be involved in structure stabilization, such as, residues that have an impact on correct folding (e.g., cysteines involved in disulfide bridges), necessary conformation change and allosteric communication with a functional site, and residues involved in posttranslational modifications, may be identified as “key residues”. To further decrease the probability to reduce or abolish function during the sequence design process, residues that are known to be involved in any desired function or affect a desired attribute, may be identified as key residues. Positions occupied by key residues are regarded as unsubstitutable positions and are fixed as the amino acid that occurs in the original polypeptide chain.

The term “key residues” refer to positions in the designed sequence that are defined in the rules as fixed (invariable), at least to some extent. Sequence positions which are occupied by key residues constitute a part of the unsubstitutable positions.

Information pertaining to key residues can be extracted, for example, from the structure of the original polypeptide chain (or the template structure), or from other highly similar structures when available. Exemplary criteria that can assist in identifying key residues, and support reasoning for fixing an amino-acid type or identity at any given position, include:

For antibodies, key residues may be selected within about 5-8 Å from the epitope region, and in some embodiments, within about 8 Å of the epitope region. It is noted that the shape and size of the space within which key residues are selected is not limited to a sphere of a radius of 5-8 Å; the space can be of any size and shape that corresponds to the sequence, function and structure of the original protein. It is further noted that specific key residues may be provided by any external source of information (e.g., a researcher).

When the template structure, the PSSM file (which is based on the full MSA and any optional context specific sub-MSA), and the identification of key residues, unsubstitutable positions and the substitutable positions are provided, the method presented herein can use these data to provide the modified antibody sequence chain starting from the original antibody sequence.

As noted above, once a set of substitutable positions and their corresponding amino acid alternatives has been determined, a position-specific stability scoring is determined for each alternative. In some embodiments, for each alternative, including the original amino acid at that position, the position-specific stability scoring is determined by subjecting a single substitution variant of the template structure (SSVTS), differing from the initial template structure by having the alternative amino acid in place of the original amino acid, to a global energy minimization, as this term is defined herein, and the difference in total free energy (ΔG), binding affinity (ΔΔG), or any other property such as aggregation propensity with respect to that of the (refined) template structure is recorded as the position-specific stability scoring for that amino acid alternative.

In some embodiments, the position-specific stability scoring is determined by subjecting the SSVTS to a local energy minimization. In such embodiments, which are advantageous in the sense of computational costs, the position-specific stability scoring is determined for each amino acid alternative, including the original amino acid at that position, by defining a weight fitting shell around the position within which all residues are subjected to a local energy minimization (weight fitting within the weight fitting shell) to determine the lowest energy arrangement for each amino acid within the shell. In case a position within the shell is occupied by a key residue, the key residue is not subjected to amino acid substitution refinement, and is subjected only to small range energy minimization without repacking. In some embodiments, the weight fitting shell has a radius of about 5 Å; however, other sizes and shapes of weight fitting shells are contemplated within the scope of the method presented herein.

According to some embodiments of the present invention, the local energy minimization is effected for amino acid residues of the modified polypeptide chain having at least one atom being less than about 5 Å from at least one atom of the position-specific amino acid alternative, thereby defining a 5 Å weight fitting shell. According to some embodiments, the weight fitting shell is defined as a 6 Å shell, a 7 Å shell, an 8 Å shell, a 9 Å shell or a 10 Å shell, while greater shells are contemplated within the scope of some embodiments of the present invention.

Thus, at least one position-specific amino acid alternative for each of the substitutable positions are identified. According to some embodiments of the present invention, the method presented herein includes a step that determines which of the positions in the amino-acid sequence of the original polypeptide chain will be subjected to amino-acid substitution and which amino acid alternatives will be assessed (referred to herein as substitutable positions), and in which positions in the amino acid sequence of the original polypeptide chain the amino-acid will not be subjected to amino-acid substitution (referred to herein as unsubstitutable positions). As noted above, flexibility or permissiveness is permitted in the rules for selection of suitable/unsuitable positions as well as the selections of mutations at those positions, in order that mutations having none or a negative effect on the predictive binding or folding energy may in combination with one or more other mutations having none, a negative, or a positive effect result in a polypeptide with improved properties.

In the single position scanning step, a position-specific stability score is given to each of the allowed amino acid alternatives at each substitutable position (see definition of substitutable positions herein above). A comprehensive list of amino acid alternatives that have a position-specific stability score below 1 r.e.u. (i.e., are predicted to be stabilizing) is referred to herein as the “sequence space”. This list is used as input for the later method step, which includes a combinatorial generation of all, or some, of the possible sequences (designed sequences), using all or some of the position-specific amino acid alternatives.

It is noted that the detailed description of the method presented herein is using some terms, units and procedures with are common or unique to the Rosetta™ software package, however, it is to be understood that the method is capable of being implemented using other software modules and packages, and other terms, units and procedures are therefore contemplated within the scope of the present invention.

According to some embodiments of the present invention, the step of determining the amino-acid alternatives which can substitute the amino-acid at each of the substitutable positions in the amino acid sequence of the original polypeptide chain, is referred to herein as “single amino acid sequence position scanning”, or “single position scanning”. This step of the method, according to some embodiments of the present invention presented herein, is carried out by individually scanning each of the predefined amino acid alternatives at each of the substitutable positions in the original polypeptide chain, using the PSSM scores as described hereinabove. The single position scanning step is conducted in order to determine which amino acid alternatives are suitable per each scanned substitutable position, by determining the change in free energy (e.g., in Rosetta energy units, or r.e.u) upon placing each of the amino-acid alternatives at the scanned position. The rate at which free energy or binding affinity is changed is correlated to a stability score or binding score, which is referred to herein as “a position-specific scoring”.

A substitutable position is defined by:
i. not being a key position;
ii. having at least one amino acid alternative that has a PSSM score equal to, or greater than a predefined cutoff; and
iii. satisfying a predefined distance cutoff to a ligand (if present).

At each substitutable position, only amino acids that have a higher PSSM score than the predefined cutoff (e.g., equal to or greater than 0), are subjected to the single position scanning step. This sequence-based restriction, together with restrictions resulting from key residues (functional), and the distance restriction typically reduces the scanning space from all positions in the sequence to a fewer positions, and further reduces the scanning space at each of these positions from 20 amino acid alternatives to about 1-10 alternatives. The single position scanning step iterates over the polypeptide chain positions while skipping key residues and unsubstitutable positions, and for each substitutable position it iterates only over the amino acid alternatives that have a PSSM score which is higher than the predefined cutoff to determine their position specific score.

For example, in some positions, the original amino acid is conserved such that that all other amino acid alternatives receive an extremely negative PSSM score which is lower that the cutoff, leading to a sampling space of 1; as a result, this position will no longer be considered substitutable. In other positions the sequence alignment shows greater variability, meaning that this position is not conserved; however, even for such positions the variability of possible amino acid ranges from about 1 to 10, as indicated by the PSSM score, and not all 20 amino acid alternatives.

The next step of the method presented herein, according to embodiments of the present invention, is generating every combination of the aforementioned identified amino acid variants at each position in the antibody sequence. This could generate easily up to 10′8 sequences that require subsequent computation evaluation for affinity and/or stability to identify potential candidates with improved properties. Depending on the number of processors available to handle this large number of sequences, a parallel computation strategy can potentially decrease the computation time by 1000- to 5000-fold. By adjusting the thresholds for passing candidate sequences from the PSSM-generated variant enumeration step to the affinity/stability evaluation step to identifying promising improved candidates for experimental evaluation, more variants can be screened and the potential to identify significantly enhanced properties by this computational affinity maturation method can readily generate candidates and avoid experimental affinity maturation altogether.

As noted above, during the combinatorial step only amino acid alternatives that passed the given acceptance threshold are allowed to permute at the corresponding substitutable positions. Noting that as previously described, flexibility is introduced into the selection process to include neutral or slightly unfavorable mutations that may in combination with other mutations provide a benefit to the sequence, for each such position only amino acid alternatives that have a position-specific stability scoring meeting these criteria are sampled combinatorially. All other residues are subjected only to repacking and conformational free energy minimization. The combinatorial step yields a final variant with a combination of mutations that are all compatible with one another.

In order to computationally determine the improvement in affinity or stability, an exemplary energy minimization procedure, according to some embodiments of the present invention, is the cyclic-coordinate descent (CCD), which can be implemented with the default all-atom energy function in the Rosetta™ software suite for macromolecular modeling. For a review of general optimization approaches, see for example, “Encyclopedia of Optimization” by Christodoulos A. Floudas and Panos M. Pardalos, Springer Pub., 2008.

According to some embodiments of the present invention, a suitable computational platform for executing the method presented herein, is the Rosetta™ software suite platform, publicly available from the “Rosetta@home” at the Baker laboratory, University of Washington, U.S.A. Briefly, Rosetta™ is a molecular modeling software package for understanding protein structures, protein design, protein docking, protein-DNA and protein-protein interactions. The Rosetta software contains multiple functional modules, including RosettaAbinitio, RosettaDesign, RosettaDock, RosettaAntibody, RosettaFragments, RosettaNMR, RosettaDNA, RosettaRNA, RosettaLigand, RosettaSymmetry, and more.

Weight fitting, according to some embodiments, is effected under a set of restraints, constrains and weights, referred to as rules. For example, when refining the backbone atomic positions and dihedral angles of any given polypeptide segment having a first conformation, so as to drive towards a different second conformation while attempting to preserve the dihedral angles observed in the second conformation as much as possible, the computational procedure would use harmonic restraints that bias, e.g., the Cα positions, and harmonic restraints that bias the backbone-dihedral angles from departing freely from those observed in the second conformation, hence allowing the minimal conformational change to take per each structural determinant while driving the overall backbone to change into the second conformation.

In some embodiments, a global energy minimization is advantageous due to differences between the energy function that was used to determine and refine the source of the template structure, and the energy function used by the method presented herein. By introducing minute changes in backbone conformation and in rotamer conformation through minimization, the global energy minimization relieves small mismatches and small steric clashes, thereby lowering the total free energy of some template structures by a significant amount.

In some embodiments, energy minimization may include iterations of rotamer sampling (repacking) followed by side chain and backbone minimization. An exemplary refinement protocol is provided in Korkegian, A. et al., Science, 2005 May 6; 308(5723):857-860.

As used herein, the terms “rotamer sampling” and “repacking” refer to a particular weight fitting procedure wherein favorable side chain dihedral angles are sampled, as defined in the Rosetta software package. Repacking typically introduces larger structural changes to the weight fitted structure, compared to standard dihedral angles minimization, as the latter samples small changes in the residue conformation while repacking may swing a side chain around a dihedral angle such that it occupies an altogether different space in the antibody structure.

In some embodiments, wherein the template structure is of a homologous antibody, the query sequence is first threaded on the protein's template structure using well established computational procedures. For example, when using the Rosetta software package, according to some embodiments of the present invention, the first two iterations are done with a “soft” energy function wherein the atom radii are defined to be smaller. The use of smaller radius values reduces the strong repulsion forces resulting in a smoother energy landscape and allowing energy barriers to be crossed. The next iterations are done with the standard Rosetta energy function. A “coordinate constraint” term may be added to the standard energy function to “penalize” large deviations from the original Ca coordinates. The coordinate constraint term behaves harmonically (Hooke's law), having a weight ranging between about 0.05-0.4 r.e.u (Rosetta energy units), depending on the degree of identity between the query sequence and the sequence of the template structure. During refinement, key residues are only subjected to small range minimization but not to rotamer sampling.

Any of several methods may be used to calculate the change in affinity or stability. In one embodiment, for any form of energy minimization procedure, implemented in the context of embodiments of the present invention, sequence data is incorporated as part of the energy calculations. The energy function contains the standard physico-chemical energy terms, such as used in the RosettaDesign software suite, and two additional terms: one is the coordinate constraint used also at the template structure refinement (see above), and the second is a PSSM-related term, which is the PSSM score (value) multiplied by a weight factor. A PSSM-related weight factor can be determined, for example, in a benchmark study.

The difference between the PSSM score that was calculated in the step described above is different from the PSSM weight factor that is discussed here. As stated above, the energy minimization method generates a local minimum of the energy function, which takes into account multiple physicochemical properties. In this invention, we introduce two new terms to the energy function to be taken into account during energy minimization:
1. Coordinate constraint—this is basically a harmonic function that assures that the structure does not deviate much from its starting conformation/template structure. For every such deviation, there is a penalty which is proportional to the square of the difference between the modified and original values.
2. PSSM Score penalty—is multiplied by a predefined weight factor, so the energy function is biased towards mutations with a higher likelihood than the background distribution. This comes into play when comparing the final score of two structures that have two different sequences. The score of the structure with the more “conserved” sequence should be more favorable.

According to some embodiments of the present invention, the PSSM score (value) of each of the amino acid alternatives (or amino acid substitutions) is at least zero.

When using the Rosetta™ suite, of each amino acid alternative, the position-specific stability scoring is determined by calculating the total free energy of the design with respect to the template structure, and the position-specific stability scoring is expressed in r.e.u.

According to some embodiments of the present invention, the position-specific stability scoring of each of the amino acid alternatives (or amino acid substitutions) is equal or smaller than zero. It is noted that a negative ΔΔG value (position-specific stability score) means that the total free energy of a tested entity is lower than the total free energy of the reference entity, and thus the tested entity is considered “more relaxed energetically”, or more stable energetically. In the context of embodiments of the present invention, negative position-specific stability scoring is correlated with lower ΔG of folding, which typically indicate higher structure stabilization; however, in order to reduce the probability to incorporate deleterious mutations in the final designed sequence, a minimal (least negative) acceptance threshold is imposed; thereby only amino acid alternatives that have ΔΔG values lower than this acceptance threshold will be permitted into the next step of the method.

As used herein, the term “acceptance threshold” refers to a free energy difference ΔΔG value, which is used to determine if a given amino acid alternative, having a given position-specific stability scoring (also expressed in ΔΔG units), will be used in the combinatorial design step of the method presented herein.

Typically, the minimal and thus most permissive (least negative ΔΔG value) acceptance threshold can be determined in a benchmark study, such as those presented in the Examples section hereinbelow. In the presented studies it was found that a minimal acceptance threshold of −0.45 r.e.u is permissive enough to provide sufficient substitutable positions with sufficient amino acid alternatives substantially without introducing false positive substitutions. It is noted herein that the method, according to some embodiments of the invention, is not limited to any particular minimal acceptance threshold, and other values are contemplated within the scope of the invention.

The single position scanning step of the method generates a limited list of possible amino acid substitutions, referred to herein as a “sequence space”, as this term is defined hereinbelow. For each acceptance threshold the output list contains all amino acid alternatives that had a ΔΔG value (i.e. position-specific stability score) more negative than the acceptance threshold (lists from stricter thresholds are subsets of lists from more permissive thresholds). The lists serve as input for the next and final combinatorial step of the method, and each list constitutes a “sequence space”, as this term is defined hereinbelow. Briefly, a sequence space is a subset of substitutions, each predicted to improve structural stability, which is greatly reduced in size compared to the theoretical space of all possible substitutions at any given position, which is 20n, wherein 20 is the number of naturally occurring amino acids and n is the number of positions in the polypeptide chain).

The current method, in contrast to earlier methods, relies on computational assessment to overcome a theoretically unmanageable space of 20\hat{ }n. As noted above, while the earlier methods in which filtering key residues and imposing a free energy acceptance threshold may reduce the number of substitutable positions in a given sequence, such reduction ignores the non-convexity of the protein mutational landscape. If two mutations individually increase or decrease a certain preselected property of a given protein, the properties of a protein combing them is not necessarily cumulative. For this reason, the methods described here do not exclude substitutions; all possible substitutions are evaluated computationally wherein combinations that may have been avoided in prior methods are included, evaluated, and may result in improved features of the polypeptide chains that would never have been identified if such individual or combination of mutations were excluded.

In the context of embodiments of the present invention, any non-naturally occurring designed protein which is homologous to an original protein as defined herein (e.g., at least 20% or at least 30% sequence identity), and having a choice of any two or more substitutions relative to the wild-type sequence that are selected from a sequence space as defined herein, is a product of the method presented herein, and is therefore contemplated within the scope of the present invention.

It is noted herein that embodiments of the present invention encompass any and all the possible combinations of amino acid alternatives in any given sequence space afforded by the method presented herein (all possible variants stemming from the sequence space as defined herein).

It is further noted that in some embodiments of the present invention, the sequence space resulting from implementation of the method presented herein on an original protein, can be applied on another protein that is different than the original protein, as long as the other protein exhibits at least 30%, at least 40%, or at least 50% sequence identity and higher. For example, a set of amino acid alternatives, taken from a sequence space afforded by implementing the method presented herein on a human protein, can be used to modify a non-human protein by producing a variant of the non-human protein having amino acid substitutions at the sequence-equivalent positions. The resulting variant of the non-human antibody, referred to herein as a “hybrid variant”, would then have “human amino acid substitutions” (selected from a sequence space afforded for a human protein) at positions that align with the corresponding position in the human protein. In some embodiments of the present invention, any such hybrid variant, having at least 6 substitutions that match amino acid alternatives in any given sequence space afforded by the method presented herein (all possible variants stemming from the sequence space as defined herein), is contemplated and encompassed in the scope of the present invention.

Selecting those polypeptide chains wherein the change is above a preselected threshold 8. Among the combinatorial set of polypeptides designed and evaluated in the prior steps, those polypeptide(s) having the preselected property meeting or exceeding the preselected threshold for that property are identified.

Identifying at least one designed polypeptide chains having a maximal favorable change in the predefined property over that of the original polypeptide chain. From among those polypeptides identified in the prior step, those with the most desirable property may be listed or ranked in order of predicted property, such that the sequences may be reviewed for further evaluation.

Such evaluation in addition to confirming the preselected property experimentally may include ease or cost of synthesis and efficiency of expression, stability, solubility, and other characteristics that may not necessarily have been the initial desired property to be optimized, but may factor into the candidacy of a particular sequence for further evaluation. In one embodiment, the output of this step achieves the objective of the invention in generating one or more polypeptides with a predicted desired trait relative to the original sequence. In other embodiments, the one or more polypeptides are further evaluated, including in one embodiment, by expression and experimental evaluation.

Each processor is capable of evaluating a number of sequences and, using a predetermined threshold value, return to the server those sequences with the most favorable increase in affinity or stability or both. The server can then combine all of the data from the individual sequences and using a further threshold, identify those sequences with the optimal values.

The output of the protocol is a set of sequences that have a handful of mutations compared to the original design, that are meant to explore the sequence/structure space around the candidate, enabling researchers to skip the experimental affinity maturation step. After computational design is finished, the amino acids sequences are converted to DNA sequences and sent to synthesis as oligonucleotides. These oligonucleotides (˜200 bp long) are then assembled by high throughput assembly to create scFvs DNA sequences, which are transformed into yeast and screened for binding the target they were designed to bind. Screening a small library of candidate antibodies yields the desired outcome of an antibody fulfilling the criteria of affinity, specificity, and stability, suitable for clinical or research uses as described above. A threshold value for biological activity of the antibody can be established and those antibodies exceeding the threshold selected for proceeding as clinical or research candidate antibodies.

Upon completing the predefined number of computations, the server outputs a summary file that contains the sequences and their associated score.

In one embodiment, a method is provided for identifying an improved epitope-specific variant antibody sequence comprising the steps of obtaining a library of variant, epitope-specific antibody sequences that have a predicted improved affinity or stability over the original epitope-specific antibody sequence in accordance with the teachings described herein above, then experimentally evaluating the sequences by following the steps of:
1. generating DNA oligonucleotide sequences comprising the CDR3 sequences of the sequences in the library, suitable for expression in an organism in which binding activity in a display or expression system is to be assessed;
2. expressing the corresponding library of scFVs comprising the CDR3;
3. screening the display or expression system for binding of the epitope and identifying therefrom one or more CDR3 comprising an improved epitope-specific variant antibody sequence.

While methods to carry out the foregoing steps are well known in the art, the methods described in PCT/US2017/34918, published as WO2017/21049, facilitate the expression of the optimal CDR3 sequences in the context of a scFv library. In brief, the restriction ligation of an oligonucleotide having a complementarity determining region (CDR) heavy chain H3 (CDRH3) and a CDR light chain L3 (CDRL3) is provided to form a full-length antibody or its fragment on a plasmid, in order to generate an antibody library. Thus, cloning of a rationally designed antibody library based on the identification of improved antibody sequences described above, are constructed of CDRH3s and CDRL3s that are ligated together to form a short oligo that can be synthesized on a DNA chip. In a following step, restriction enzymes are used to restrict the CDRH3-CDRL3 DNA oligos so that the appropriate variable framework DNA sequences can be cloned by a 2-way ligation resulting in a full length scFV on a display or expression plasmid. In one embodiment, a recombinant nucleic acid sequence comprising: a nucleic acid sequence of complementarity determining region (CDR) of heavy chain H3 (CDRH3) and a nucleic acid sequence of complementarity determining region (CDR) of light chain L3 (CDRL3) are provided, wherein said CDRH3 is fused to said CDRL3 by a sequence comprising one or more restriction enzyme cleavage sites. The restriction enzyme cleavage sites of the invention may facilitate for cloning into a nucleic sequence of an antibody framework in order to result in a full length of an antibody or a single chain variable fragment (scFv) on an expression vector.

Thus, a display or expression plasmid library may be generated for experimentally evaluating the antibody sequences identified by the methods herein. Screening for antigen binding and selecting the sequences with characteristics that can serve as clinical or research reagent leads are then identified and pursued. As described above, expression of the antibody sequence may be a feature that can be optimized during the evaluation of the vast number of sequences generated by the methods herein.

As noted above, the methods of the invention are suited for carrying out in a server-worker computational environment, in which a server linked to multiple workers (CPUs) is suited to carry out the vast number of computations required to narrow down the enumerated sequences from the PSSM methods to the final selection of candidates for experimental evaluation. In such a manner, the computational affinity maturation function of the invention is ideally achieved.

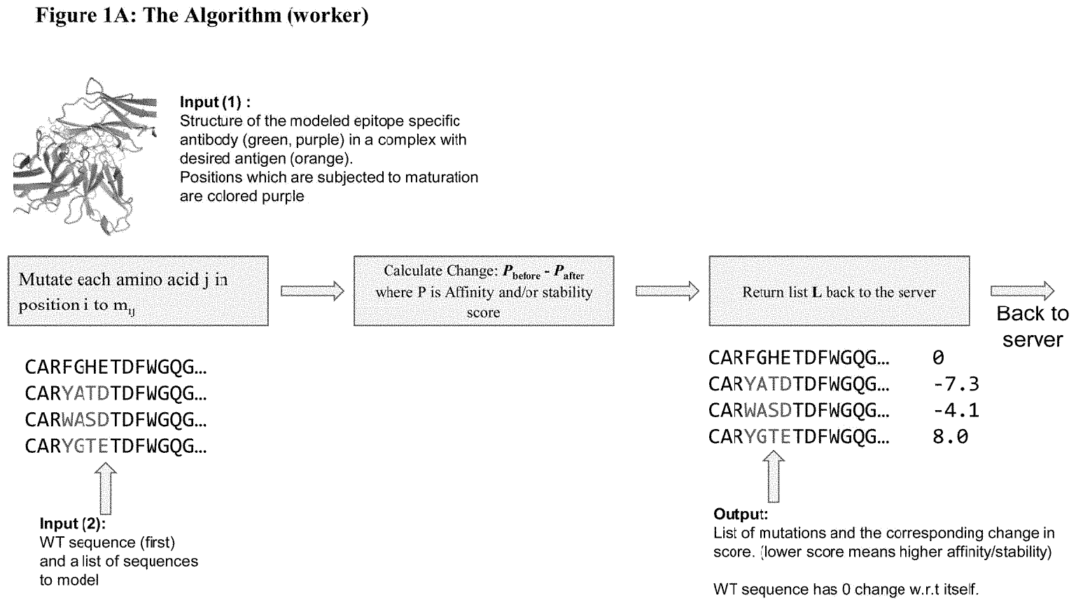

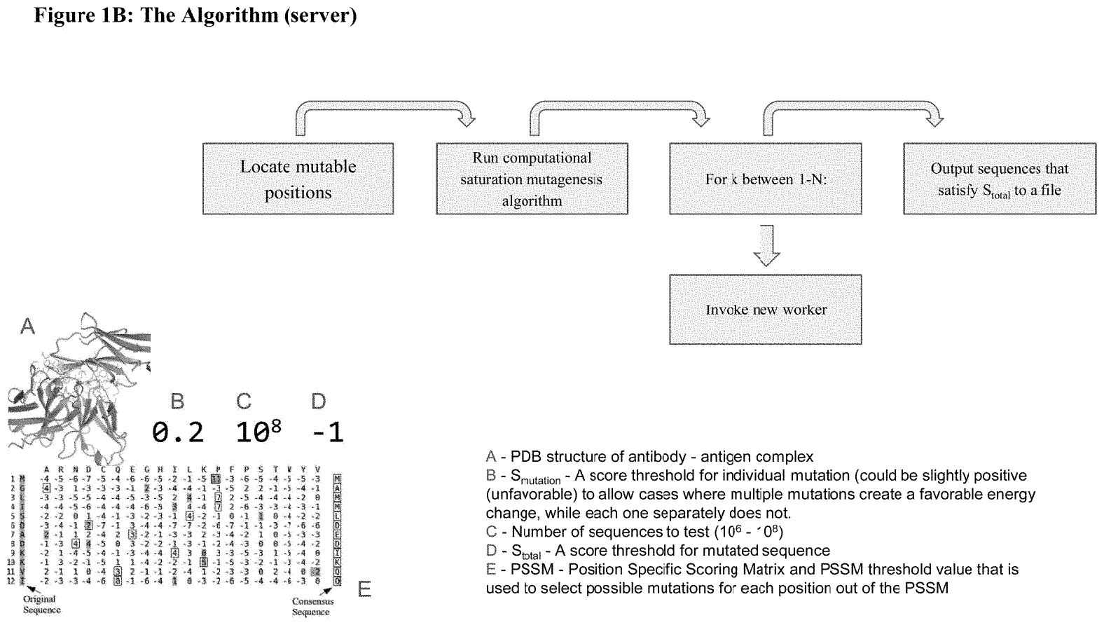

As depicted in the accompanying drawings, the following is a description of the algorithm that is run by the worker (FIG. 1A). One iteration (one sequence) takes 5 seconds on a modem CPU.

### Input:

PDB structure of antibody-antigen complex
Mᵢ—list of mutations that satisfy the given threshold (outputted from the position by position computational saturation mutagenesis from the previous step, including the WT variant)
   mᵢⱼ—mutation to amino acid j in position i
S—WT sequence

### Output

A list L of mutations and their corresponding score P. (L=[<M₁, P₁>, <M₂, P₂> . . . ])

### Flow:

1. Accept a list of mutations Mᵢ from the server

2. Mutate each amino acid j in position i to mᵢⱼ

3. Calculate change in P (P_{before}− P_{after}), where P is Affinity and/or stability score

4. Return a list L to the server

The algorithm that is run by then server (FIG. 1B).

### Input:

PDB structure of antibody-antigen complex
PSSM—Position Specific Scoring Matrix and PSSM threshold value that is used to select possible mutations for each position out of the PSSM
Sₘᵤₜₐₜᵢₒₙ—A score threshold for individual mutation
Sₜₒₜₐₗ—A score threshold for mutated sequence
N—Number of sequences to test (10⁶-10⁸)
J—Number of concurrent jobs to run

### Output:

A list L of mutations and their corresponding score P. (L=[<M₁, P₁>, <M₂, P₂> . . . ])

### Flow:

1. Find positions that should be mutated—the default strategy is to select all residues in the scFv that are within 8 Å of the antigen.
2. Run computational saturation mutagenesis algorithm, as described in Whitehead et-al: For each position i, choose from i-th row in the PSSM matrix amino acids that pass the input PSSM threshold value. Then, mutate the i-th position to that amino acid and calculate the change in P (P_{before}−P_{after}), where P is Affinity and/or stability score.
   a. If P is lower (=better) than Sₘᵤₜₐₜᵢₒₙ, insert <i,m> (m is an amino acid) to the allowed mutation list M.
3. For k between 1 and N:
   a. Create a 100 sequence enumerations of M, such that for each position -> Mₖ
   b. Invoke a new worker with:
      i. PDB Structure of the complex
      ii. Mₖ
      iii. WT sequence
      iv. Upon the worker's completion, receive list of mutated sequences and discard sequences that do not satisfy Sₜₒₜₐₗ
4. Output sequences that satisfy Sₜₒₜₐₗ to a file

One the server reports the results of candidate sequences that meet criteria for experimental evaluation, a display method can be used to determine the optimal antibody.

First, the sequences are divided the sequences to bins, based on their corresponding VH/L pairs. Then, for each bin, the amino acid sequences that are outputted by the algorithm are converted to back to DNA by back-translation. (for each amino acid, a codon is selected out of a distribution that is defined by the codon usage of the organism to which we clone the DNA molecules, in our case, it's cerevisiae). Subsequently, DNA oligonucleotides are chip-synthesized (<200 bp) of the CDR3s, while the rest of the scFv is synthesize using gene-based synthesis (Genescript/TWIST).

For each bin, Golden Gate assembly is used and, in one embodiment, the methods described in PCT/US2017/34918, published as WO 2017/21049, may be used to assemble the CDR3s and the rest of the scFv.

The scFvs are then cloned and transformed into yeast for yeast display experiments. A threshold binding activity can be utilized to select binding activity, and candidate sequences meeting the threshold selected for expression, further evaluation, and may lead to the identification of a new and useful clinical antibody or research reagent.

FIG. 1A depicts the algorithm carried out by the worker in the various embodiment described herein. An example of an antibody structure is shown (purple and green; lower left portion) bound to an antigen (orange region; upper right). The regions of the antibody subjected to the computation affinity maturation of the present invention are shown in purple, which are part of the epitope-specific region of the antibody. The worker receives as input from the server (see further below) the wild-type or original sequence, and a list of variant sequences to model. The worker, for each variant sequence, calculates the change in affinity or stability score (or both) of the value before and after variation from the original sequence; a negative result means that the variant sequence has a higher (better) score. The scores of the variants evaluated by the worker are returned to the server.

FIG. 1B depicts the algorithm carried out by the server in the various embodiments described herein. In the lower left is shown the antibody-antigen complex as described in FIG. 1A. The server identifies a value for the energy score change for each individual mutation, where a negative value indicates a favorable change (and therefore this mutation will be included in the next step) and where a positive value represents an unfavorable change wherein such mutation would not be included in the next step, except that to allow for a slight positive (slight unfavorable) change that may in combination with mutation(s) in other positions (negative or slightly positive themselves) result in a net favorable effect, mutations with slight positive scores will be passed along to the next step. The threshold for the decision to include or exclude slightly positive scores can be adjusted depending on various factors such as the number of resulting individual mutations that then need to be combinatorially enumerated and evaluated by the workers (see prior description). The matrix at the bottom left depicts these data in the PSSM used to select the possible mutations from the wild type (first column). As shown in the flow diagram, the output from this computational saturation mutational algorithm is passed to the workers for each of the sequences to be evaluated, and among these typically large number of sequences, those with the most favorable (negative Sₜₒₜₐₗ) scores provided as output from the server for identifying candidate sequences for experimental evaluation to confirm and further winnow the selections to those candidates for potential industrial or biomedical applications, as therapeutic candidates or research tools.

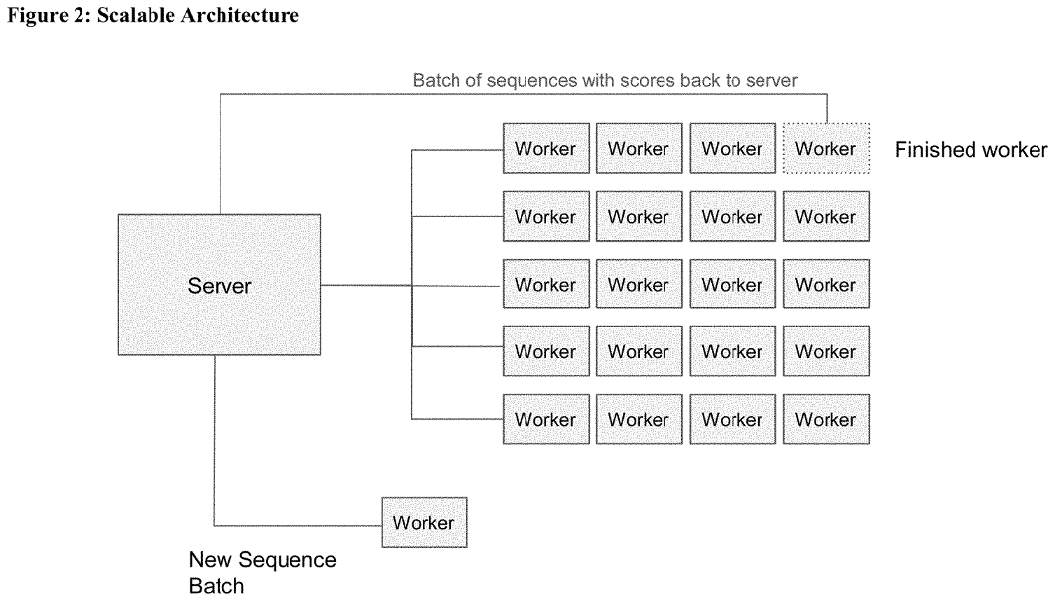

FIG. 2 depicts the organization between the server and workers in carrying out the overall algorithm of the invention. The architecture is scalable depending on the number of available workers and the number of sequences generated by the server needing evaluation by workers, and the speed at which workers can process the evaluation and proceed to the next sequence. Depending on the number of mutations, the locations of the mutations within the epitope-binding region and the interactions among the mutations in the evaluation of the score, and depending on the desired threshold for accepting variant sequences, the server can distribute the sequences for optimal evaluation by the available workers to generate a list of candidates in an acceptable period of time.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions illustrate some embodiments of the invention in a non-limiting fashion.

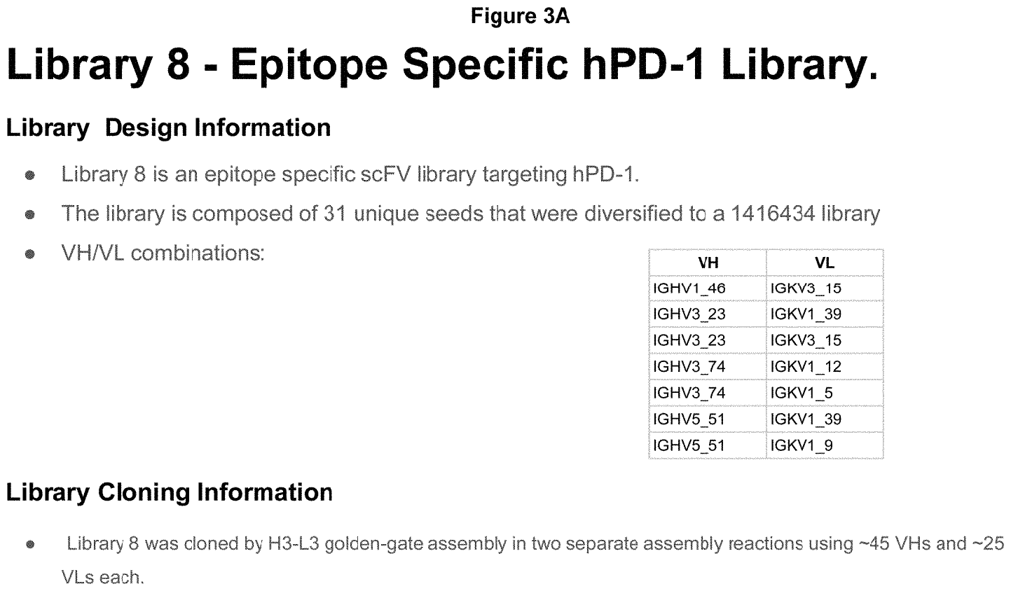

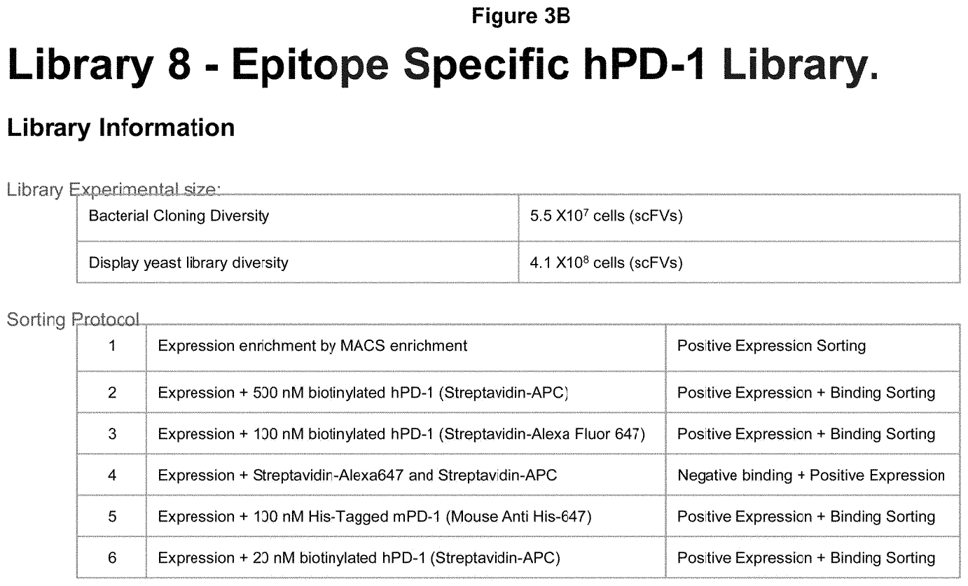

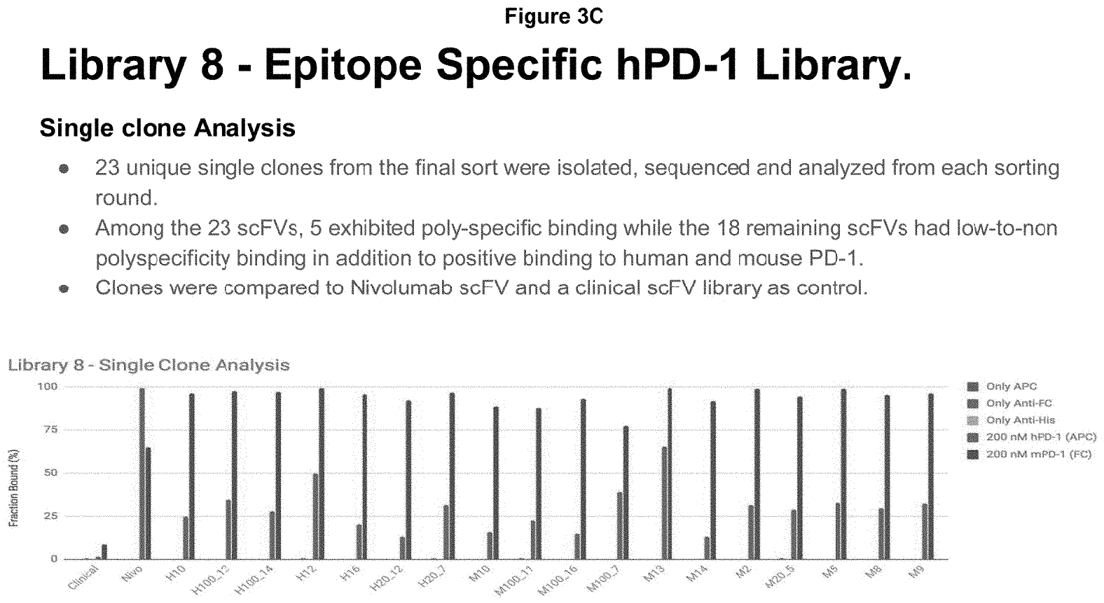

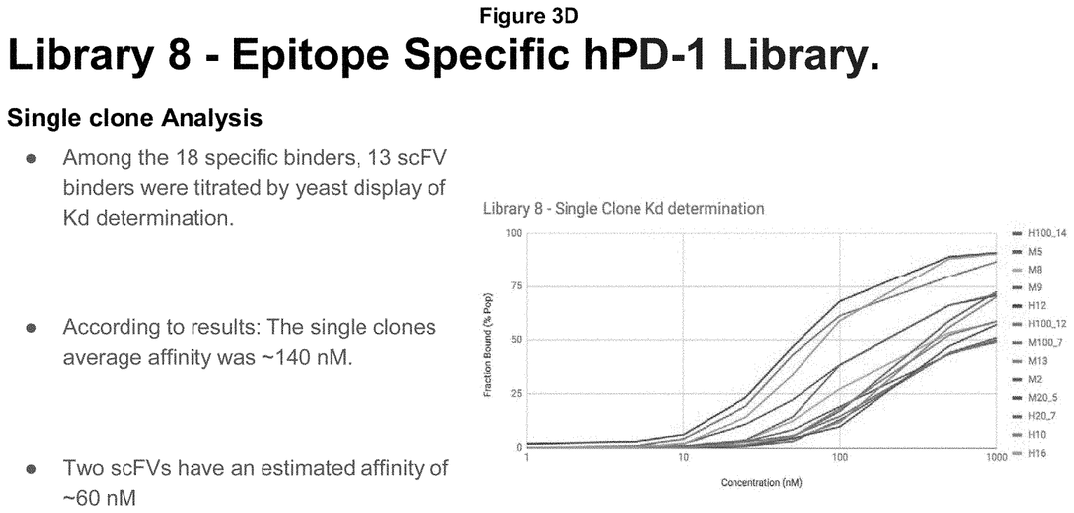

FIG. 3A-D depict an epitope specific scFV library targeting human PD-1 (hPD-1; Library 8). FIG. 3A describes the library design information and library cloning information. FIG. 3B provides library information and the sorting protocol. FIG. 3C shows the results of single clone analysis. FIG. 3D shows that among 18 specific binders, 13 scFV binders were titrated by yeast display, and the average affinity was ˜140 nM. Two scFVs have an estimated affinity of ˜60 nM.

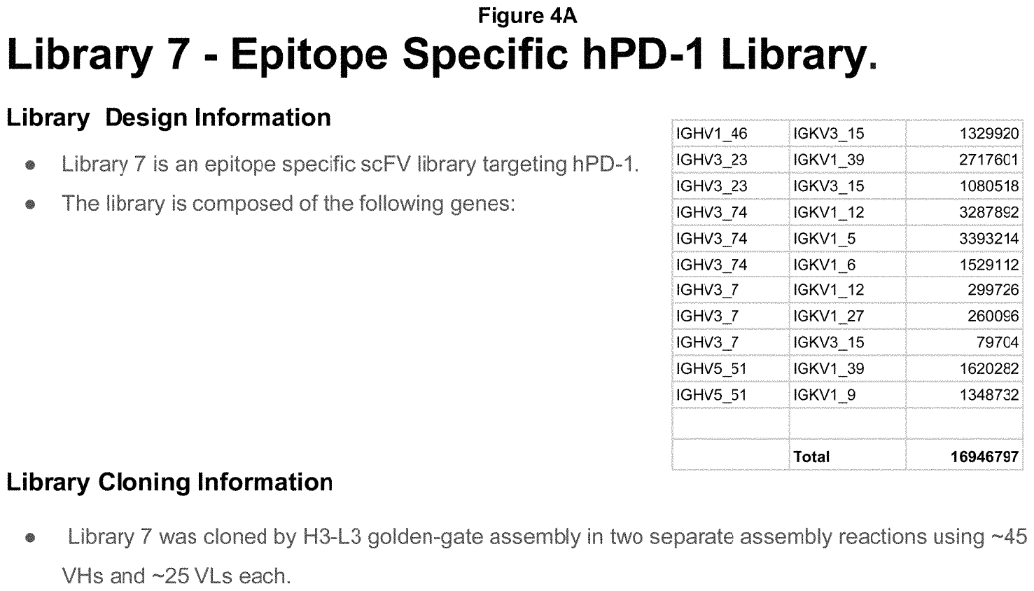

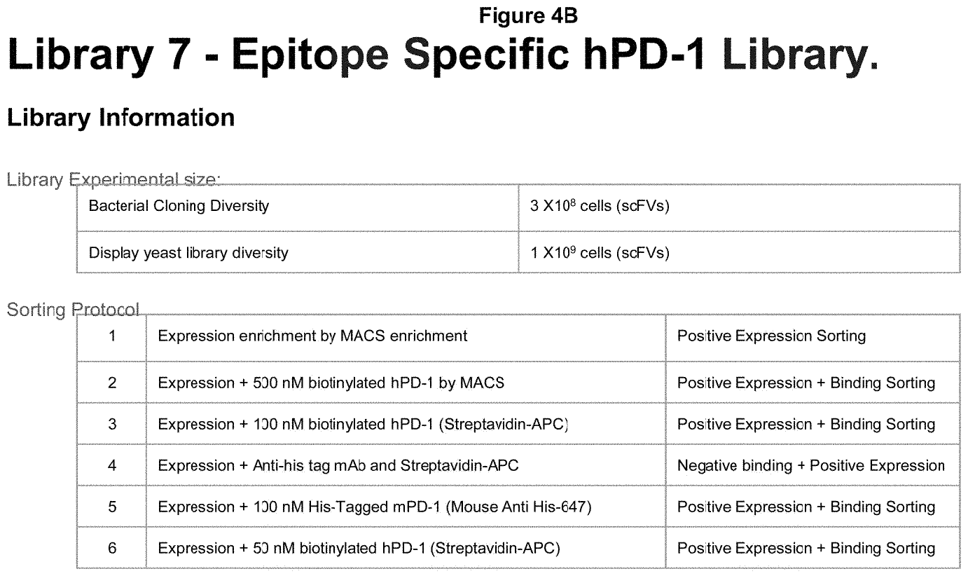

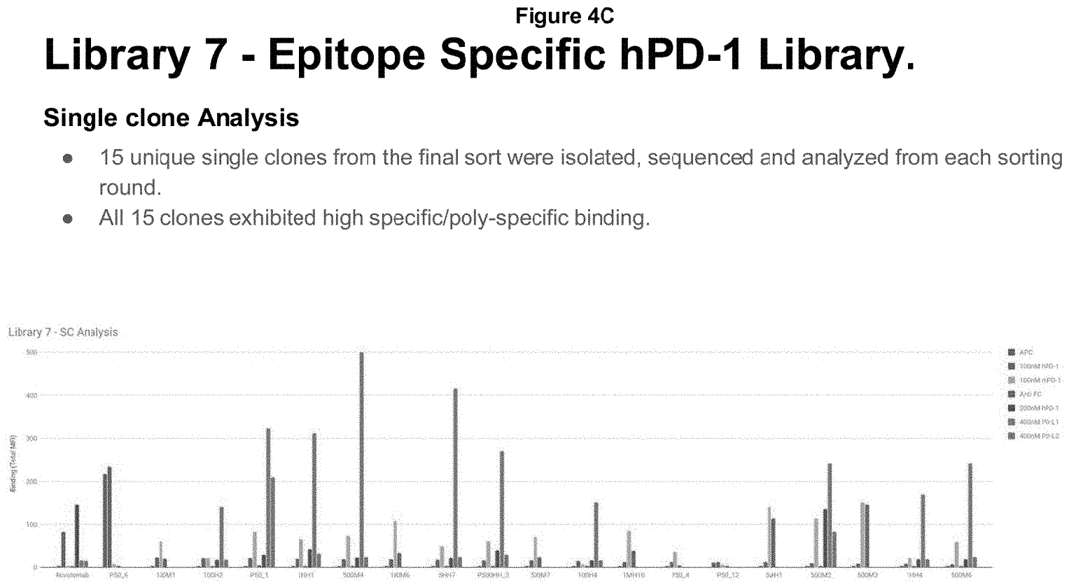

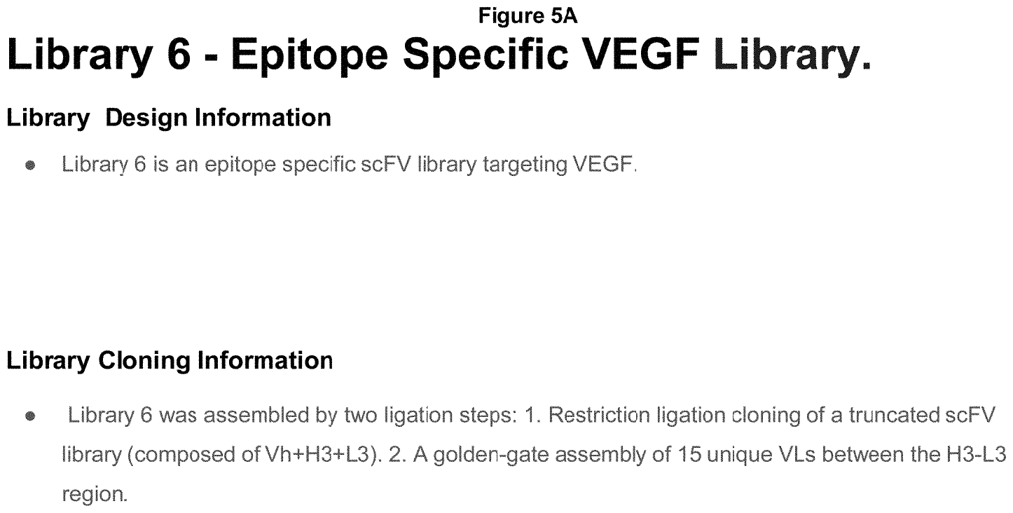

FIGS. 4A-C depict another epitope specific library targeting hPD-1 (Library 7). FIG. 4A describes the library design information and library cloning information. FIG. 4B provides library information and the sorting protocol. FIG. 4C shows the results of single clone analysis.

Fifteen unique single clones were isolated, sequences and analyzed. All 15 clones exhibited high specific/poly-specific binding.

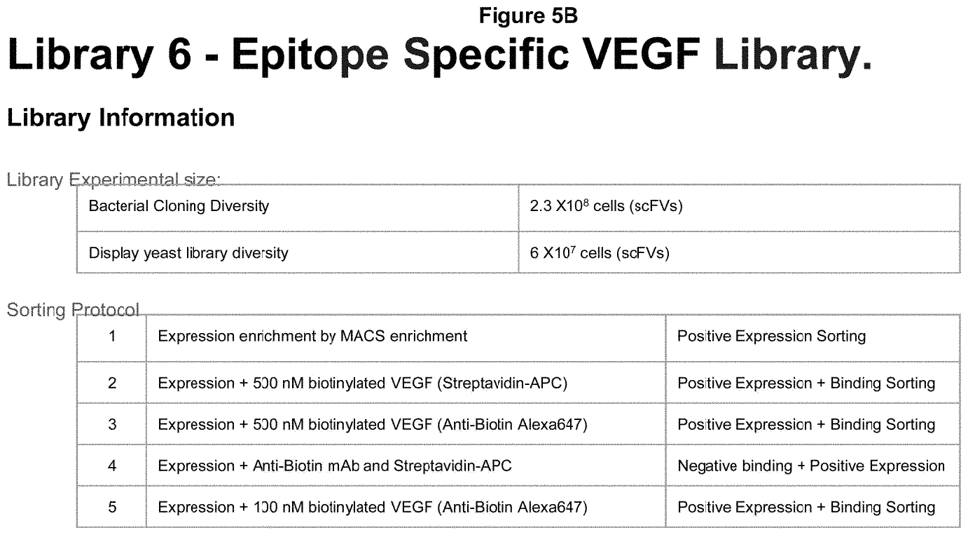

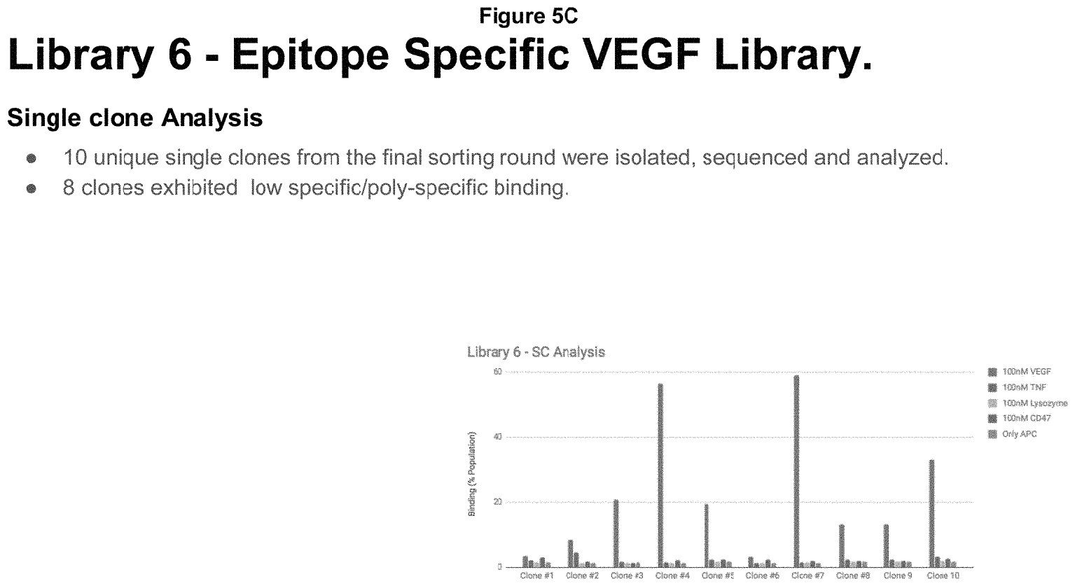

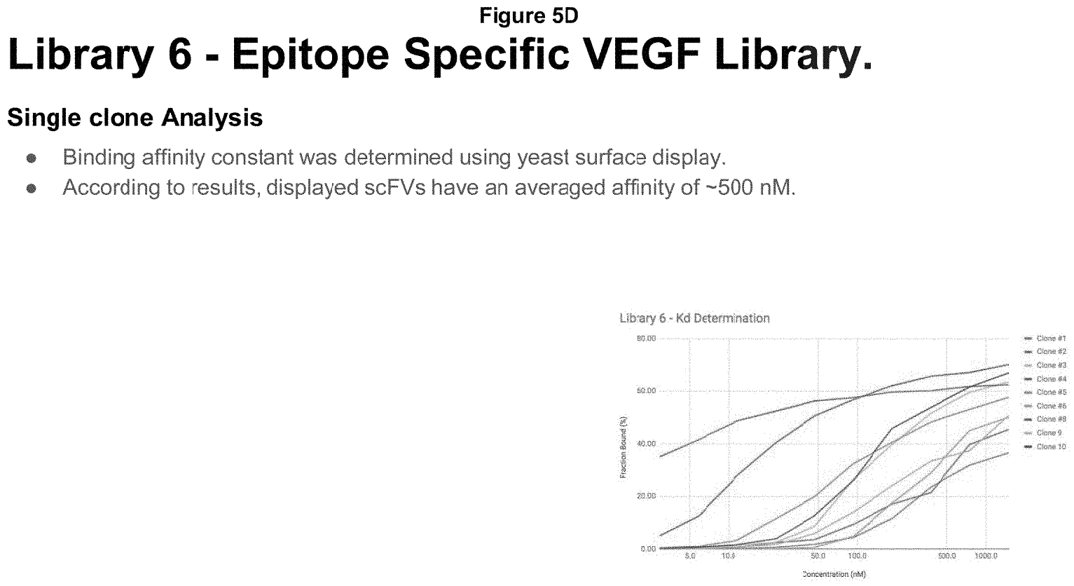

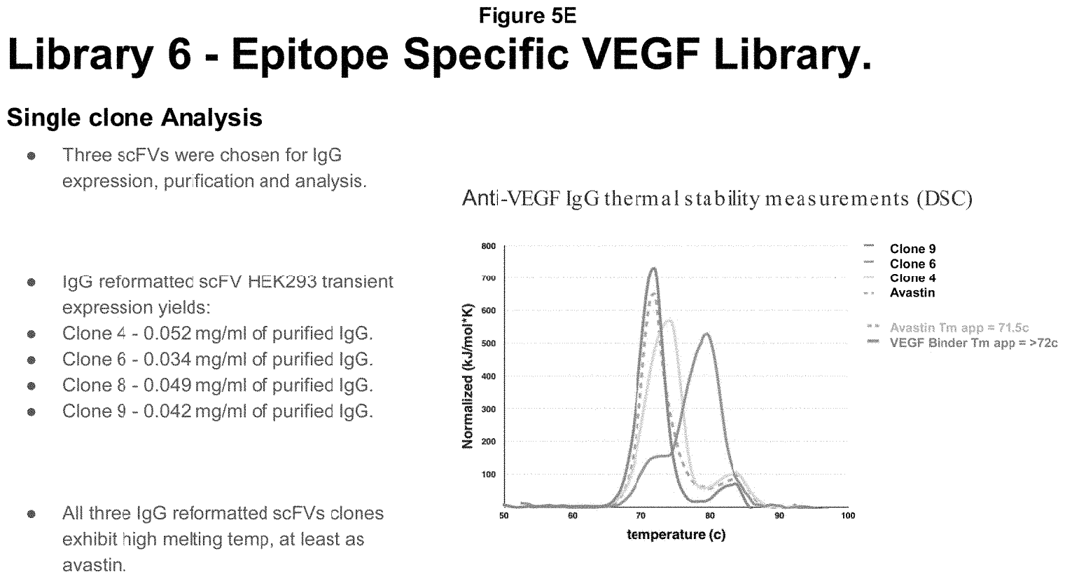

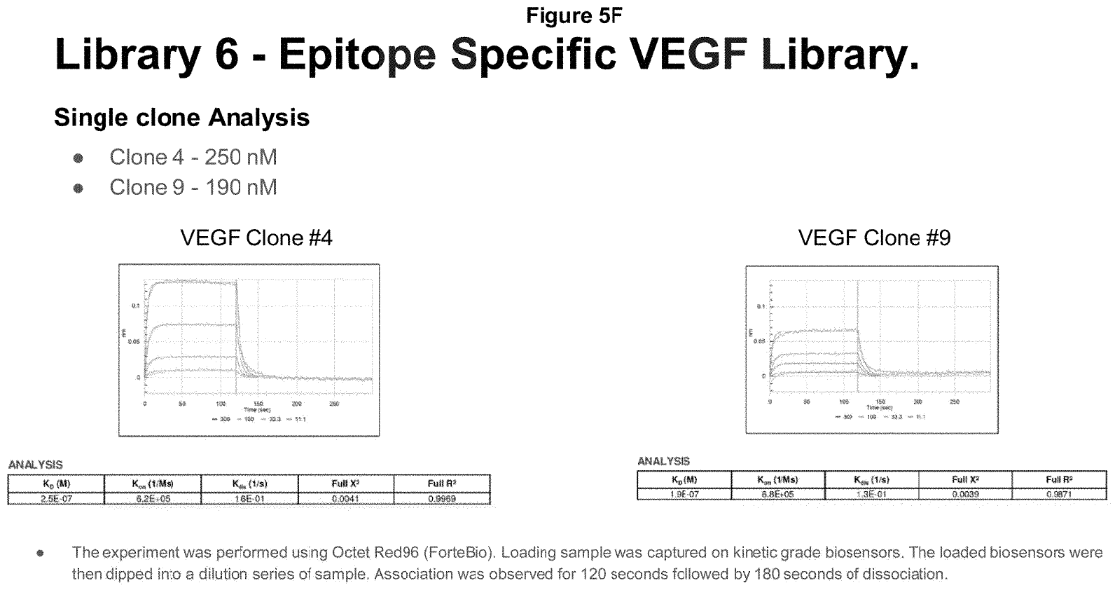

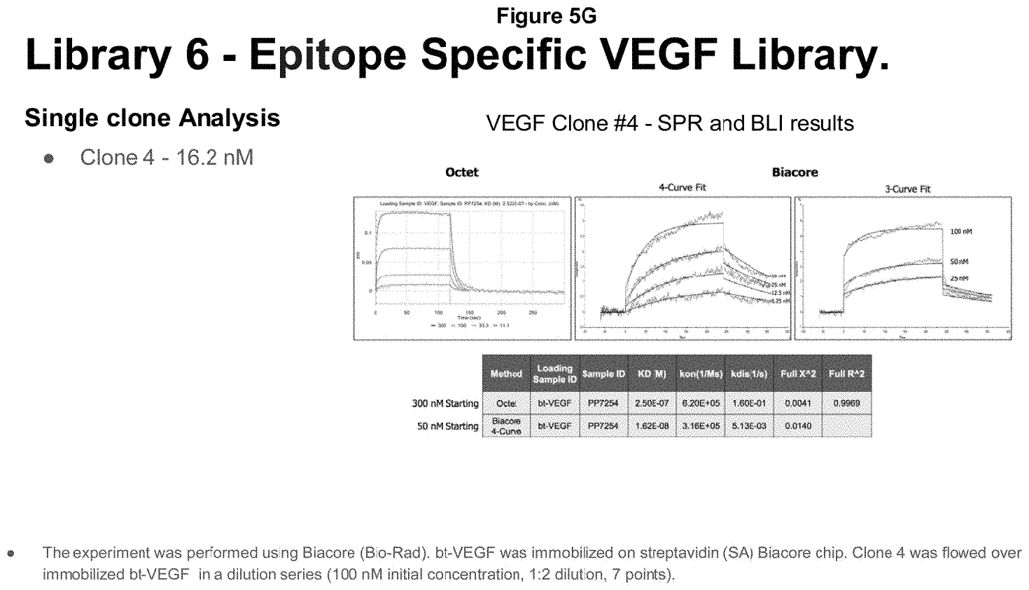

FIGS. 5A-G depict an epitope specific library targeting VEGF (Library 6). FIG. 5A describes the library design information and library cloning information. FIG. 5B provides library information and the sorting protocol. FIG. 5C shows the results of single clone analysis. FIG. 5D shows the results of single clone analysis. FIG. 5E shows the transient expression yields of clones 4, 6, 8 and 9, and the result of thermal stability determination by DSC on three clones and avastin. All three scFV clones exhibited a high melting temperature, at least as high or higher than that of avastin. FIG. 5F shows the results of binding kinetics determination of clones 4 and 9, and FIG. 5G shows binding kinetics using Biacore.

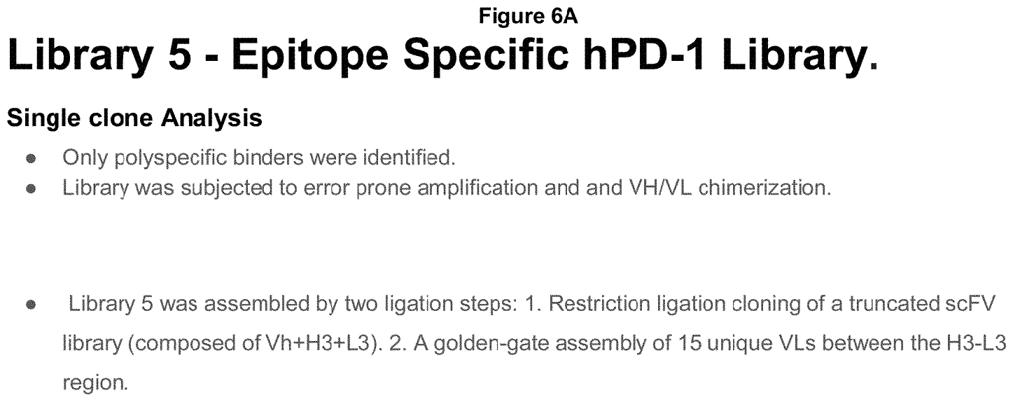

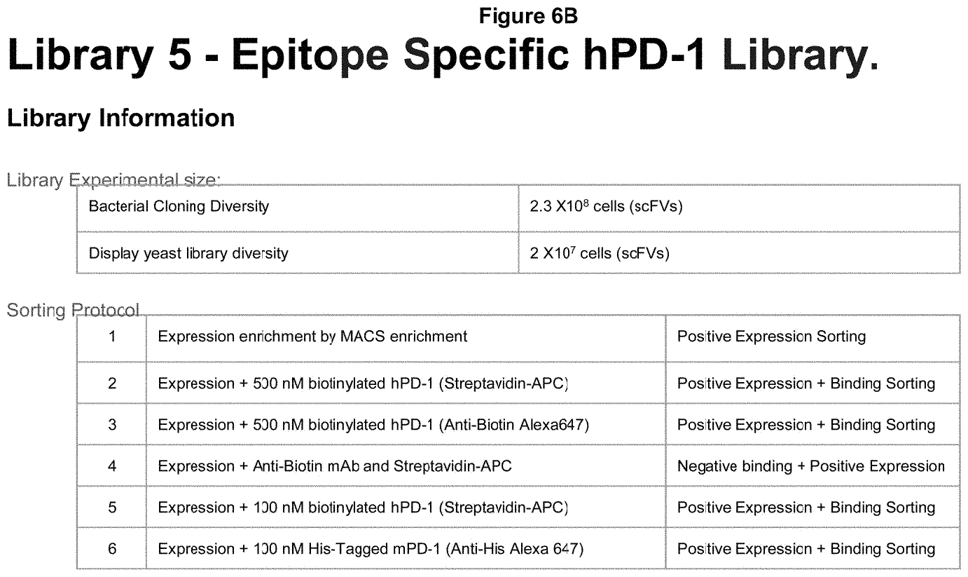

FIGS. 6A-B depict an epitope specific library targeting hPD-1 (Library 5). FIG. 6A describes the single clone analysis. FIG. 6B provides library information.

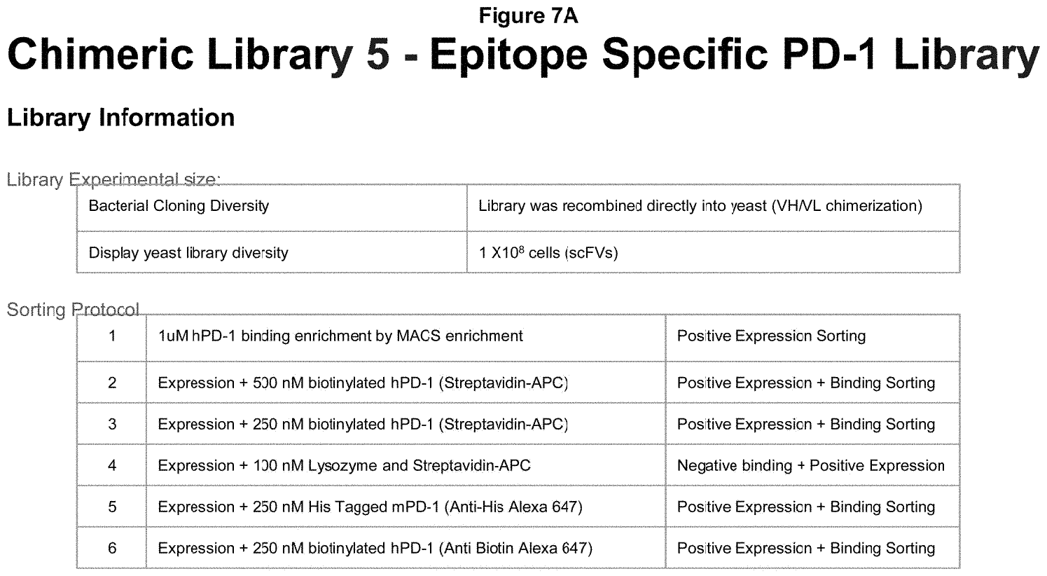

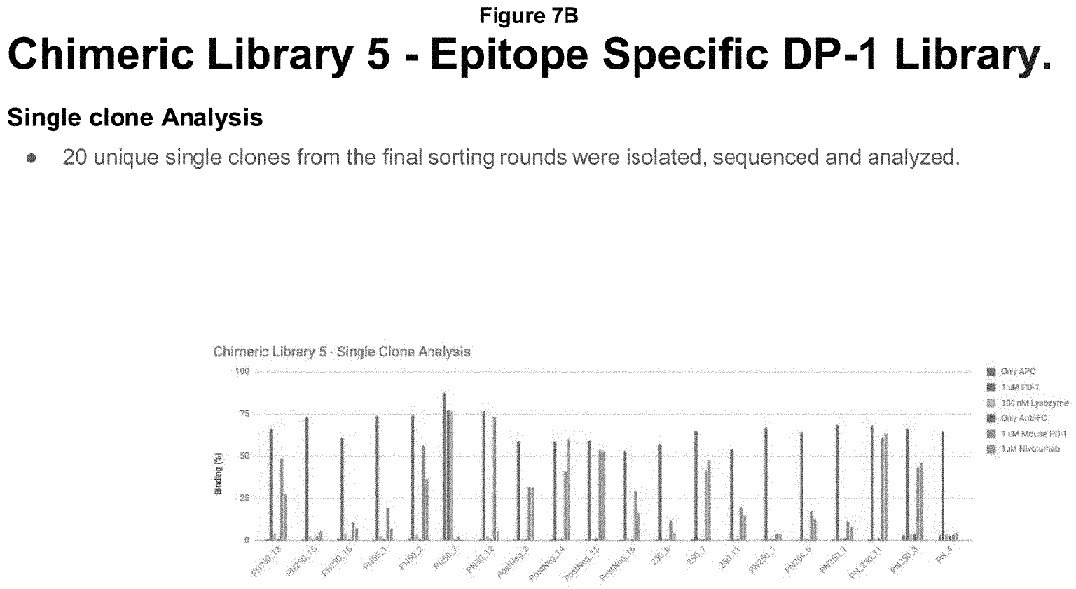

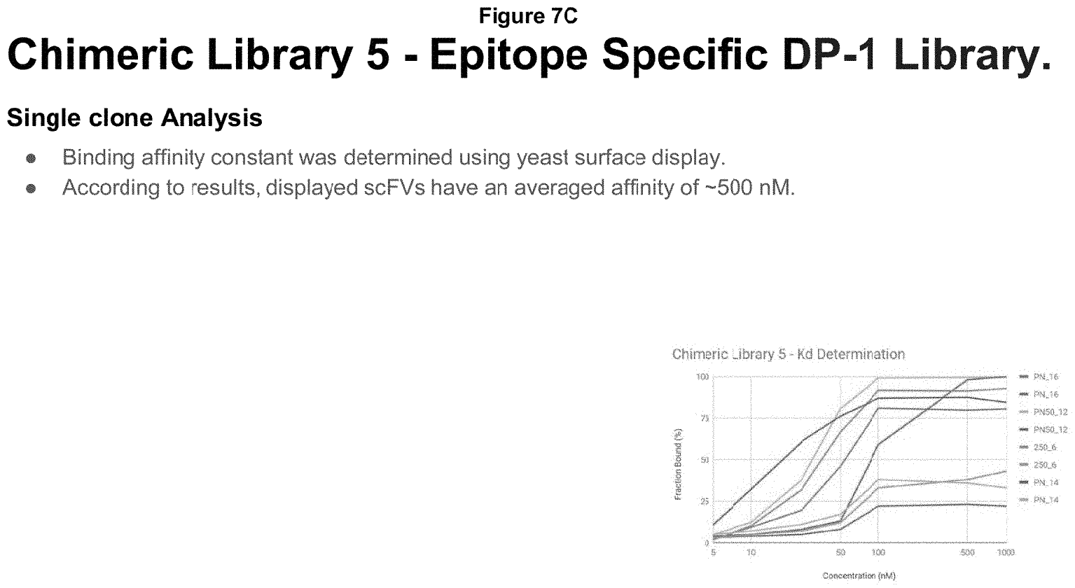

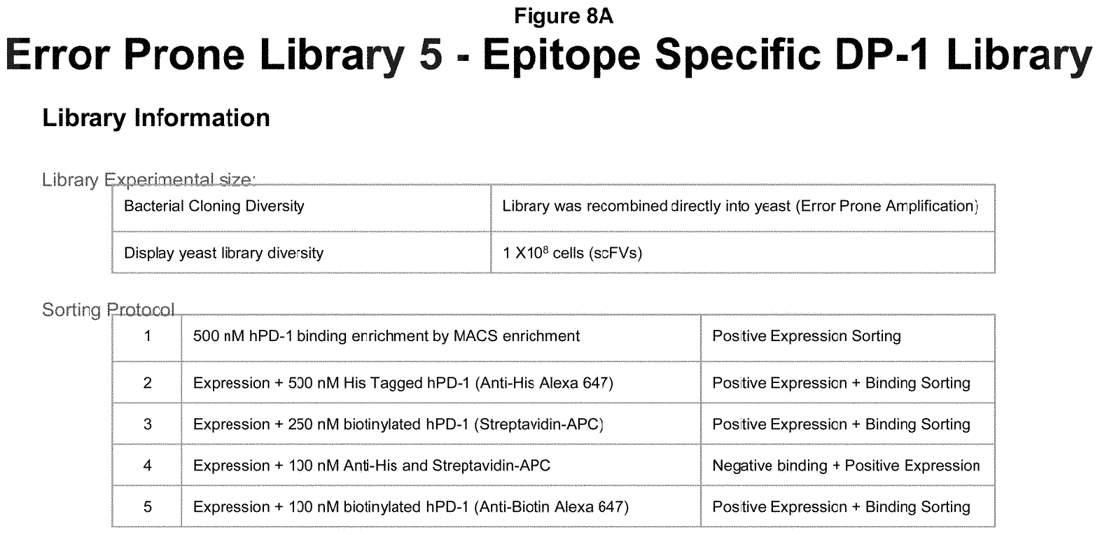

FIGS. 7A-C depict a chimeric, epitope specific library targeting PD-1 (chimeric library 5). FIG. 7A describes the library information. FIG. 7B depicts the results of single clone analysis. FIG. 7C shows the results of single clone analysis.

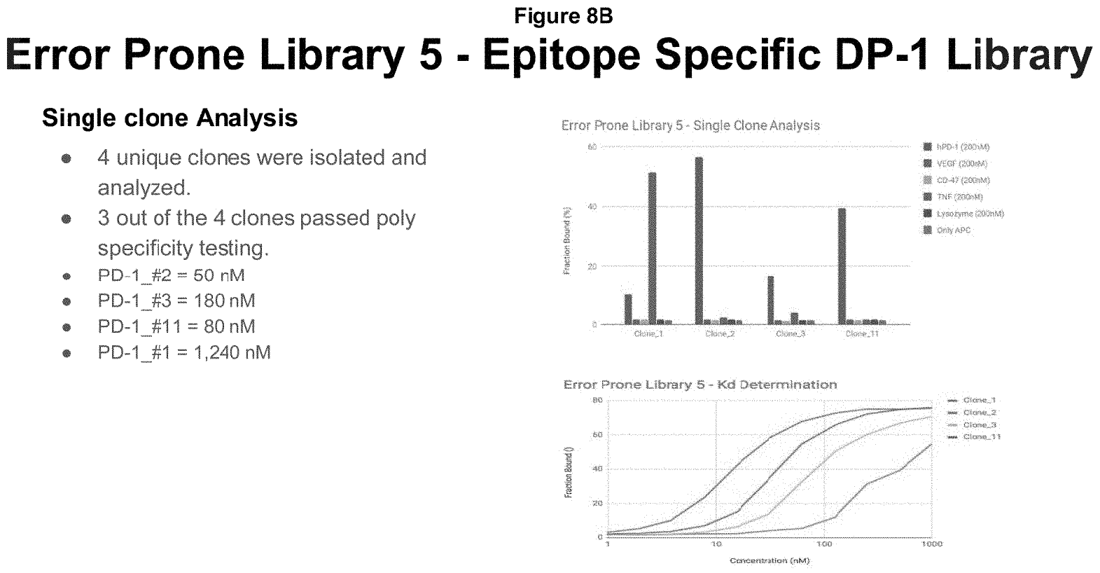

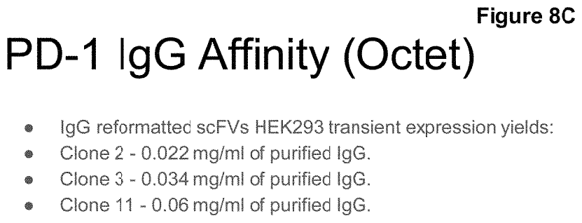

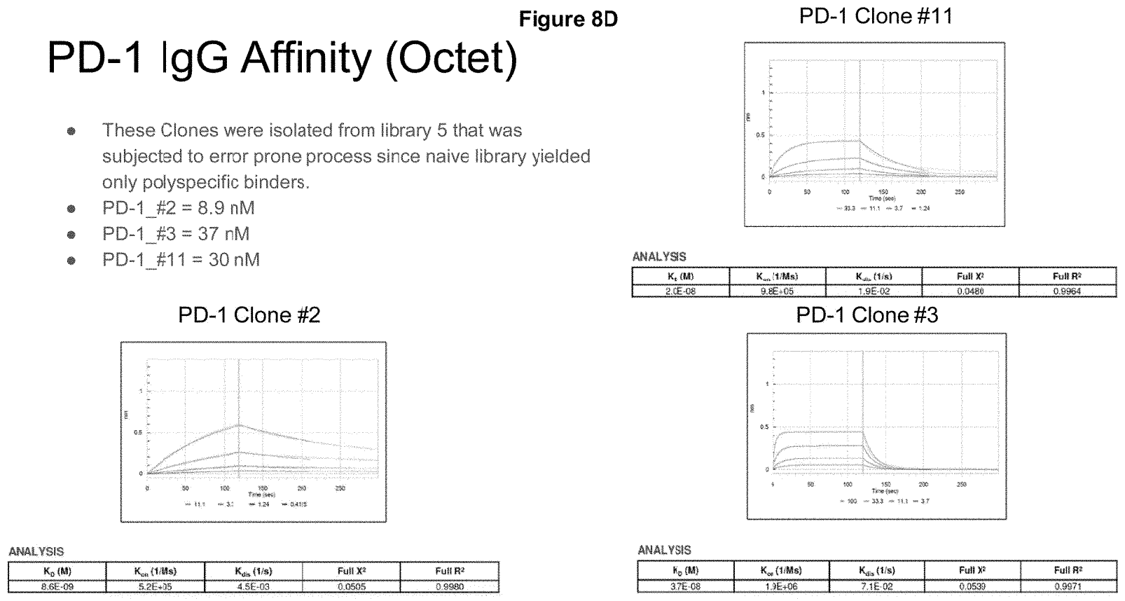

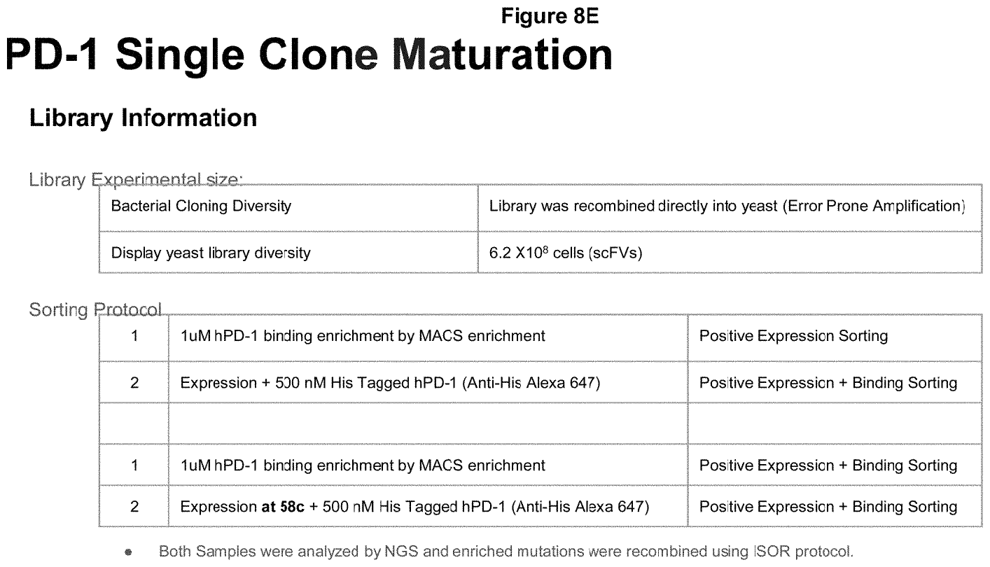

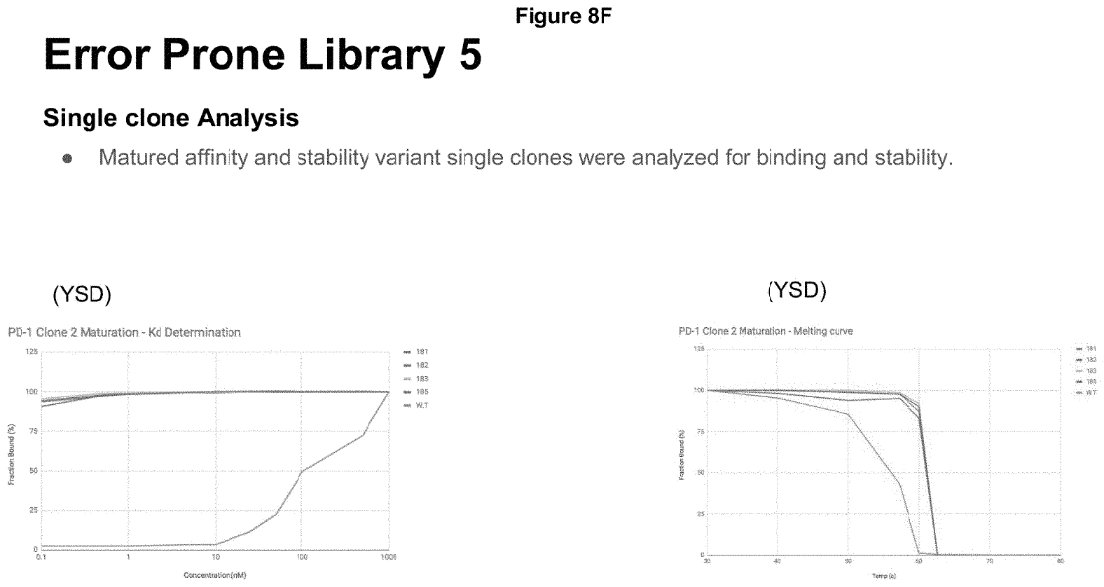

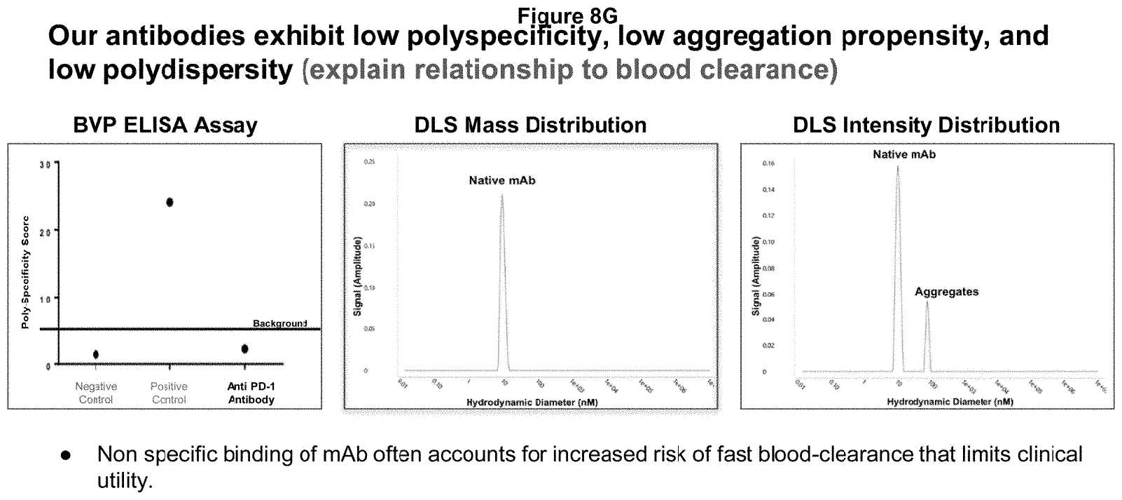

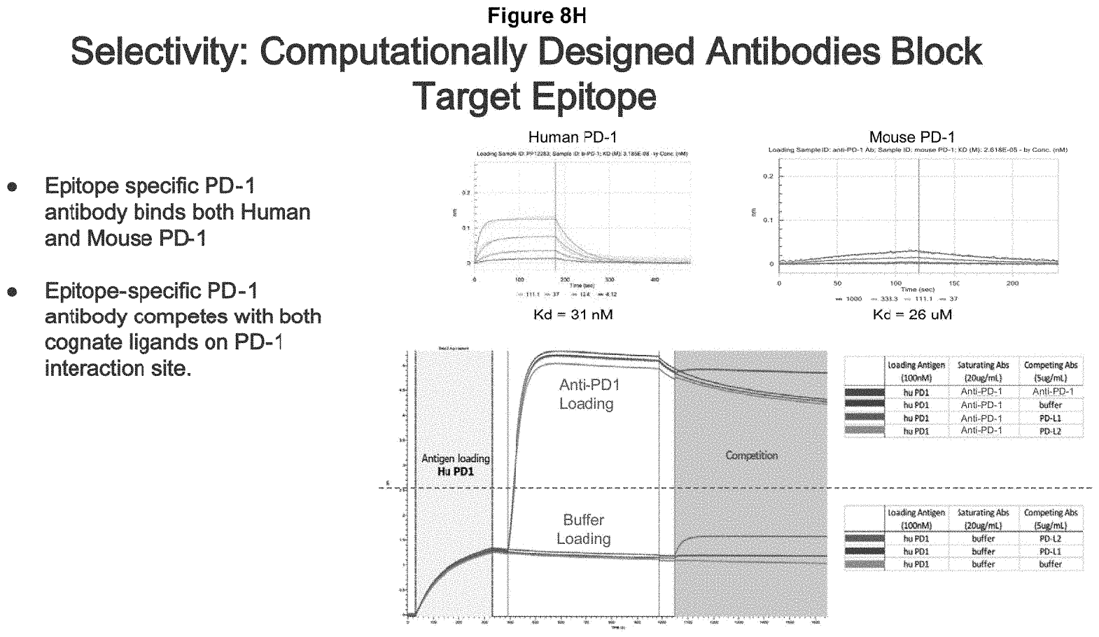

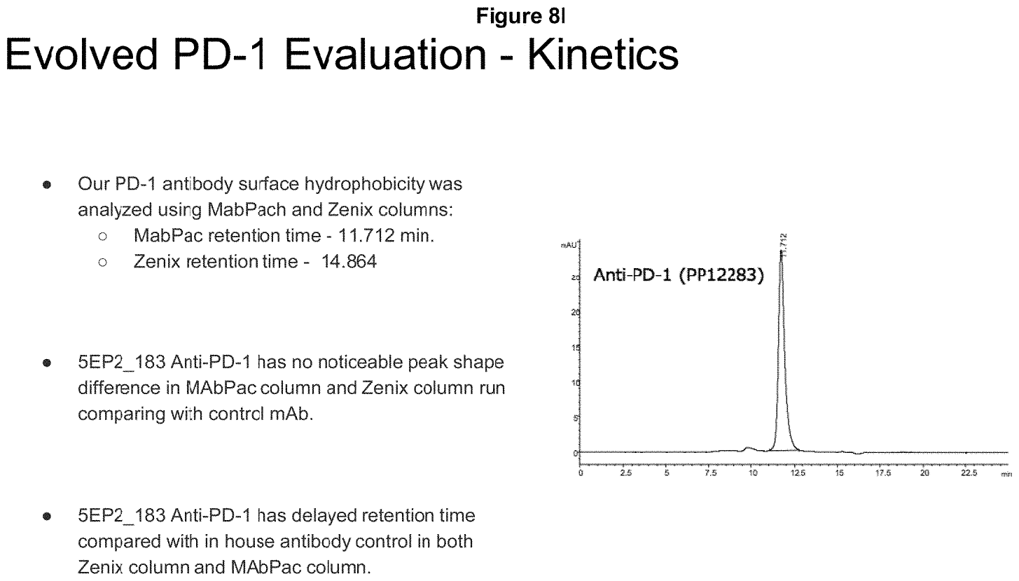

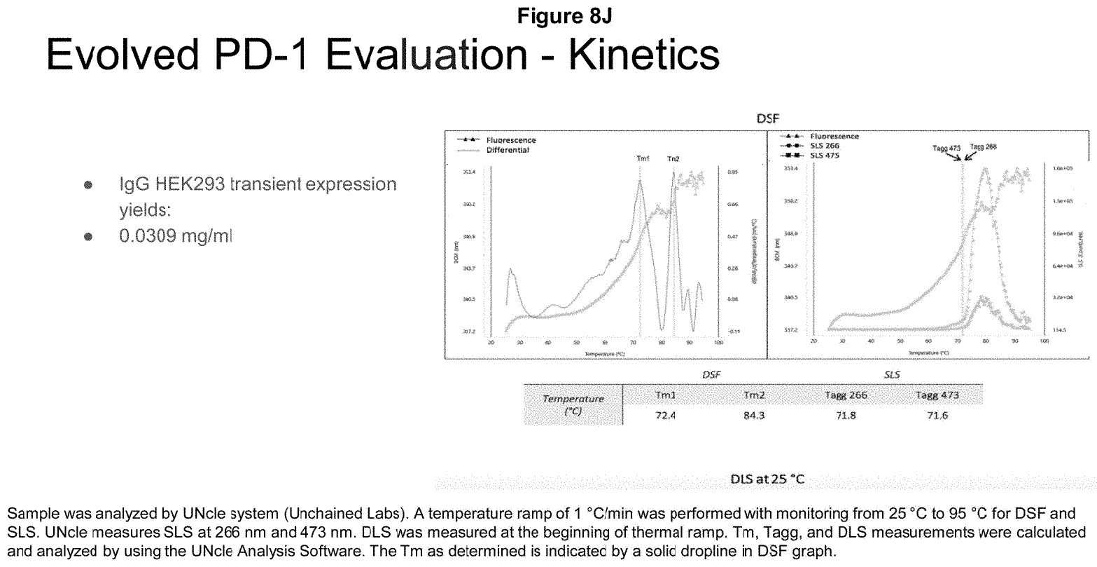

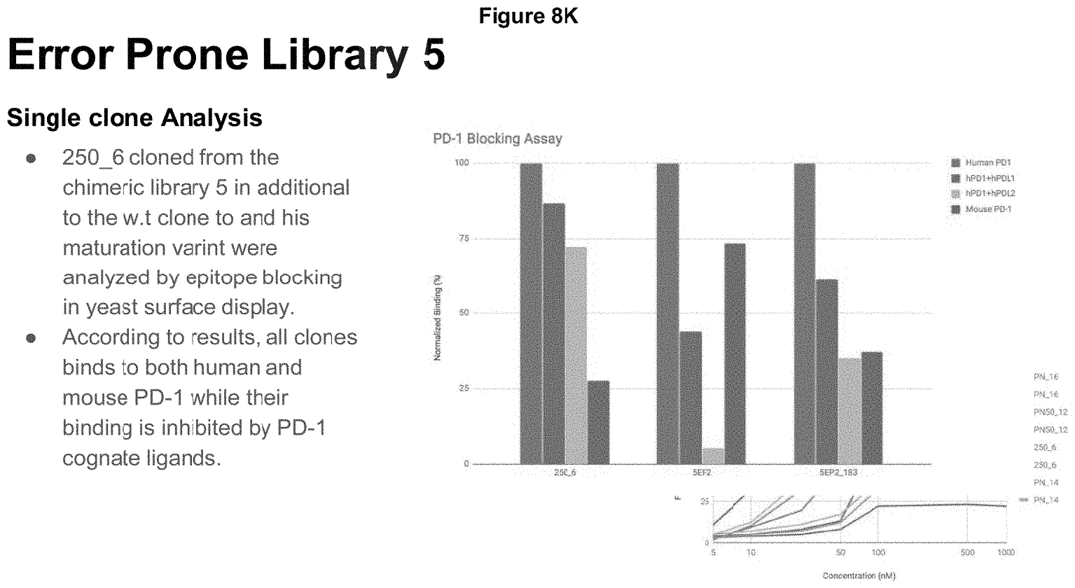

FIGS. 8A-K depict an error-prone, epitope specific library targeting PD-1. FIG. 8A shows the library information. FIG. 8B shows the results of single clone analysis. FIG. 8C shows the transient expression yields. FIG. 8D shows binding kinetics analysis of three clones. FIG. 8E shows the results of single clone maturation of clone #2: library information. FIG. 8F shows single clone analysis of matured affinity (left) and stability (right) of variant single clones. FIG. 8G shows the poly-specificity score of antibodies of the invention compared to native antibodies, the low aggregation propensity and low polydispersity. FIG. 8H shows that computationally designed antibodies block the target epitope. FIG. 8I shows a kinetic evaluation of the antibody. FIG. 8J further depicts the kinetics of the evolved anti-PD-1 antibody. FIG. 8K shows using single clone analysis that all clones bind to both human and mouse PD-1 and that binding was inhibited by their cognate ligands.

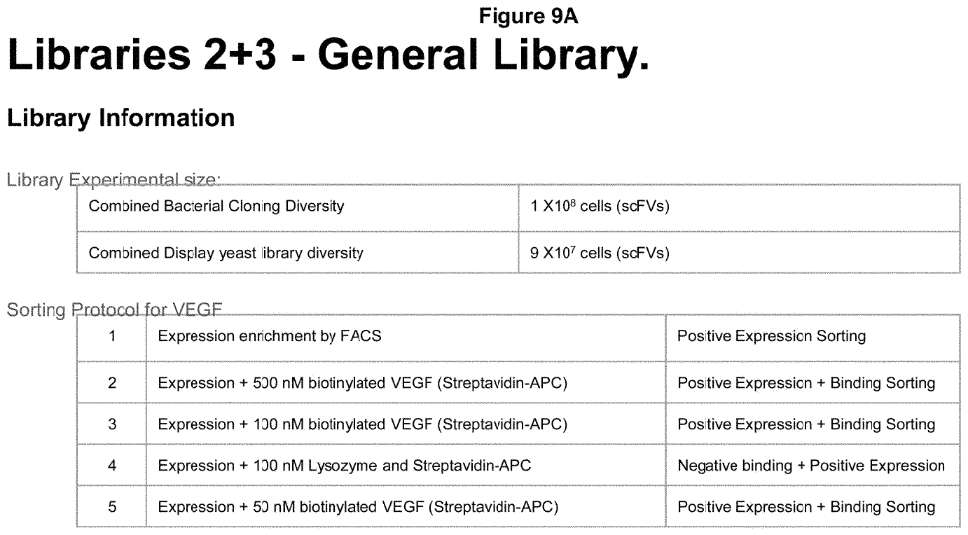

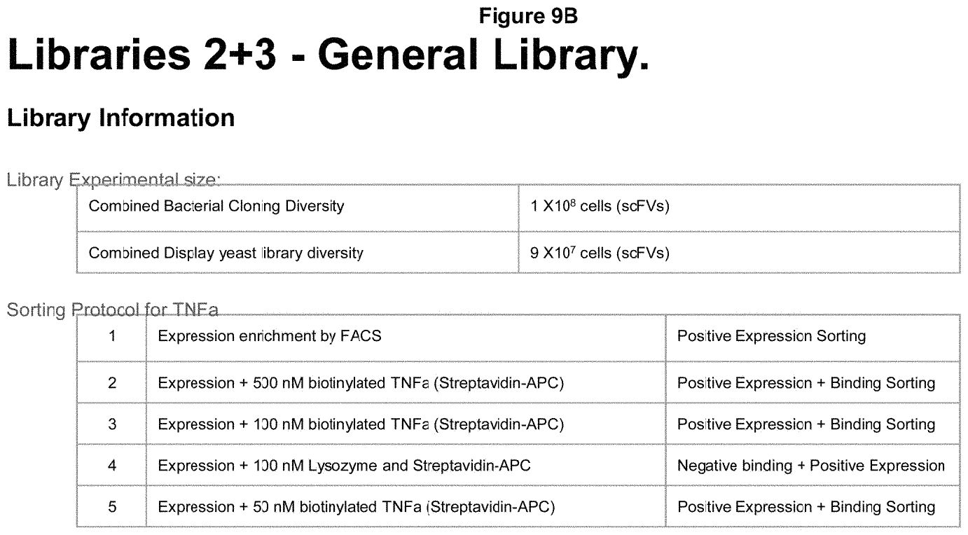

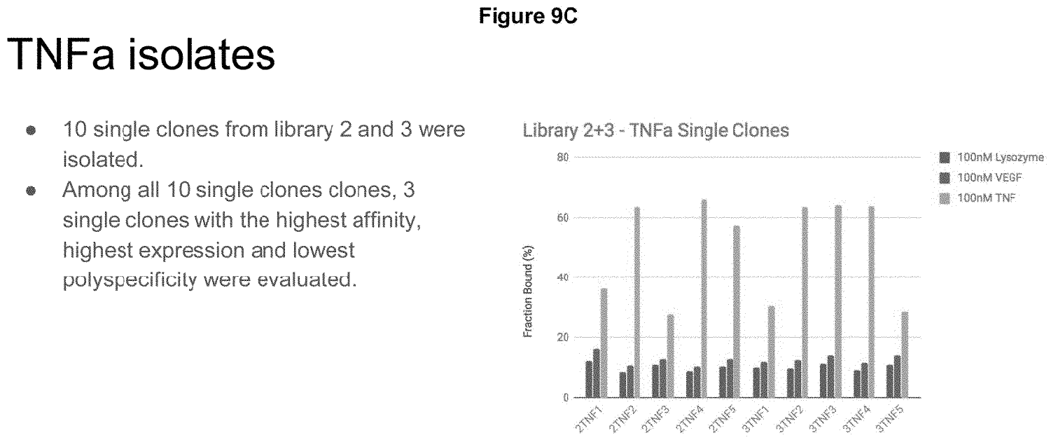

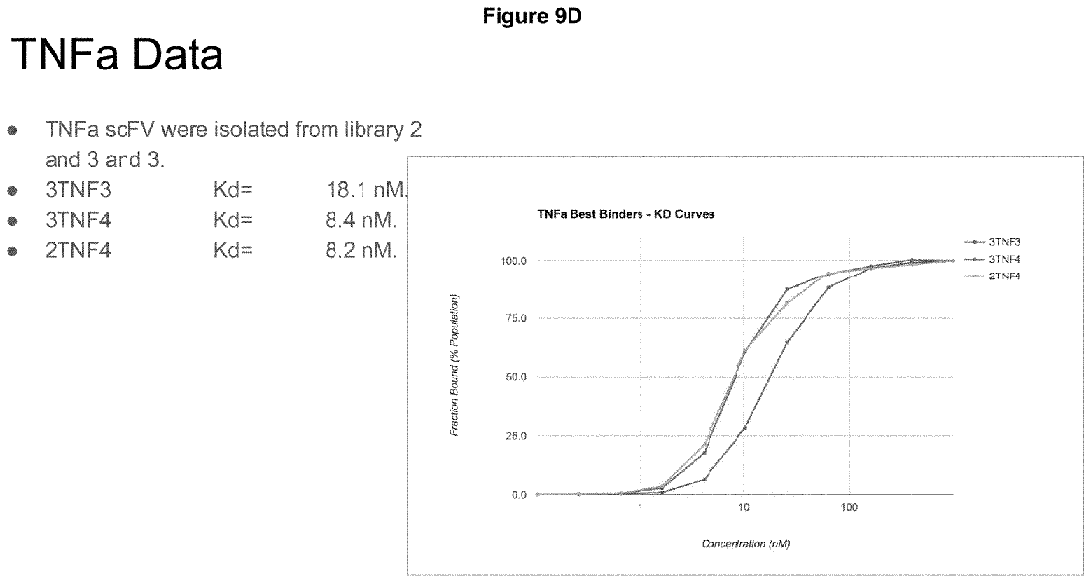

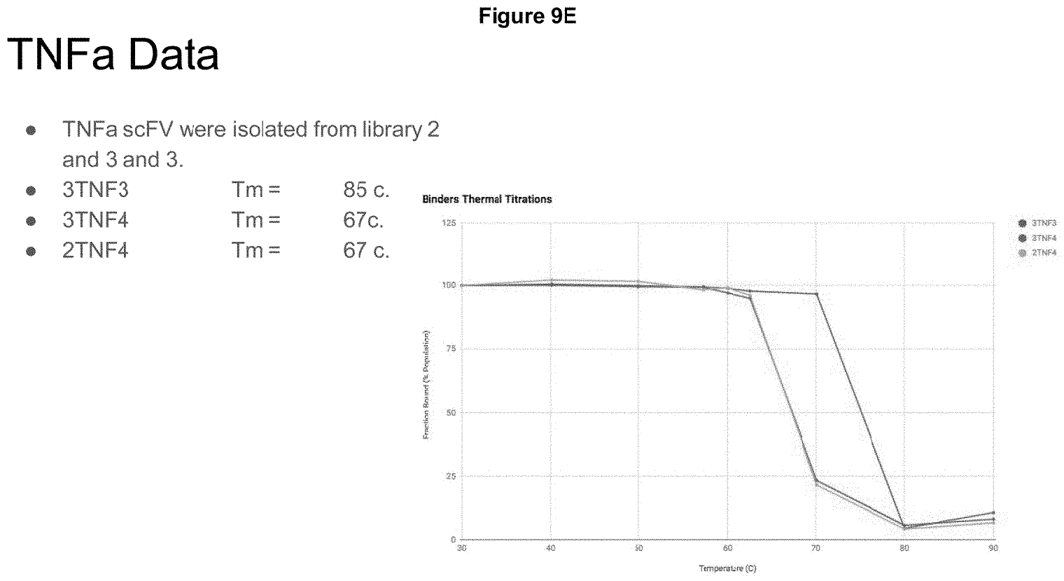

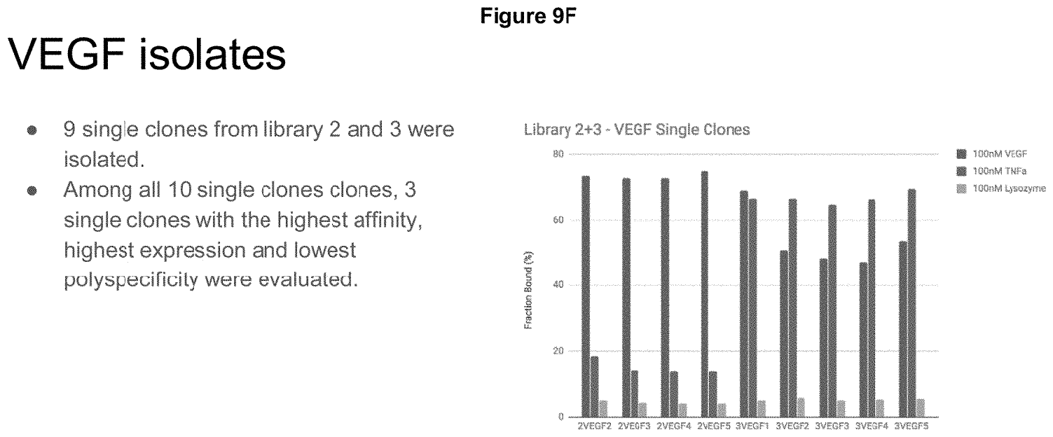

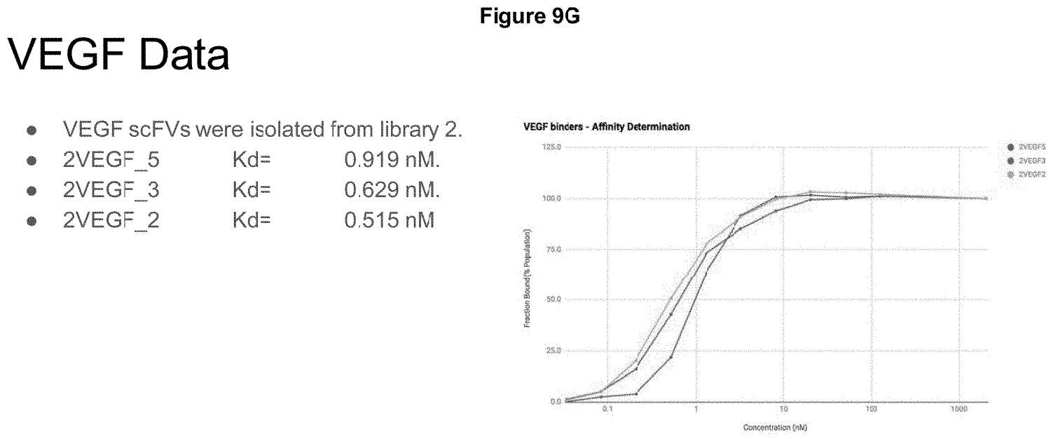

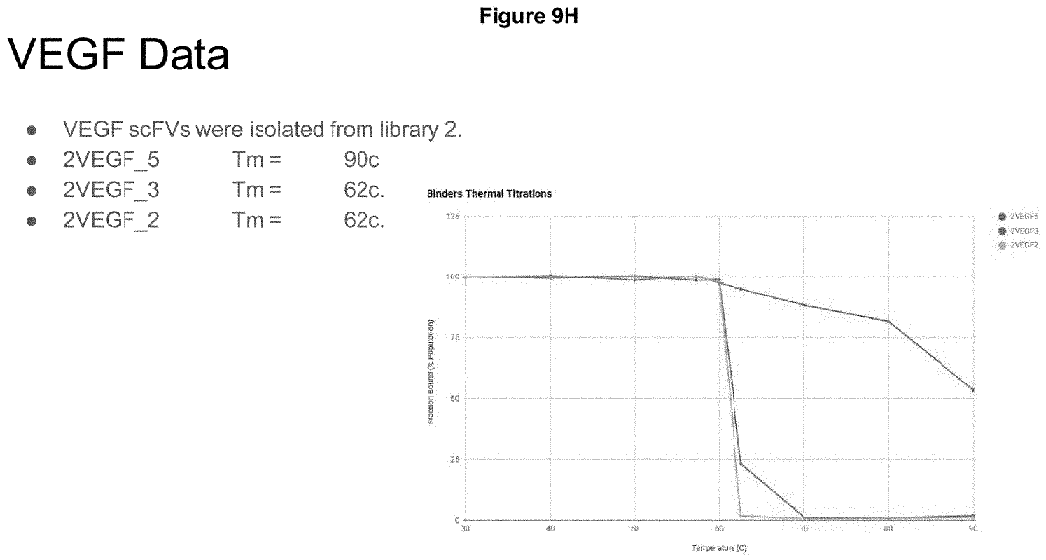

FIGS. 9A-B depict information on general libraries of antibodies against VEGF (FIG. 9A) and TNFa (FIG. 9B). FIG. 9C describes the binding activity of anti-TNFa clones. FIG. 9D depicts the Kd of the best TNFa binding antibodies. FIG. 9E shows the thermal stability of the same antibodies. FIG. 9F shows the binding activity of the anti-VEGF clones. FIG. 9G shows the Kd determination for the best anti-VEGF antibodies. FIG. 9H shows the thermal stability of the same VEGF antibodies.

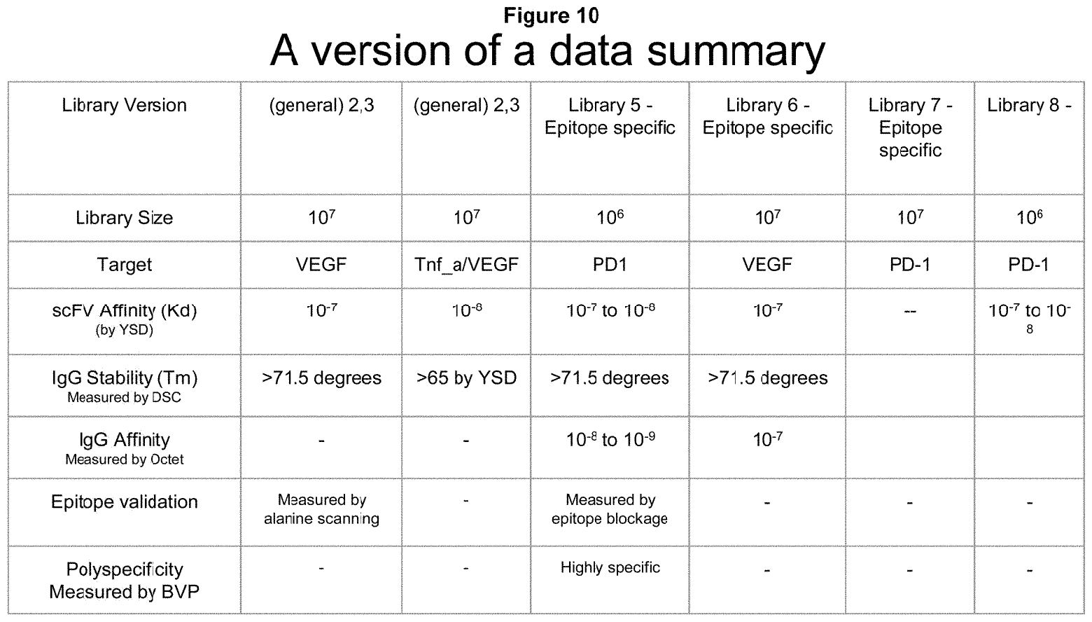

FIG. 10 summarizes the data from these examples.

What is claimed is:

1. A computer implemented method for generating a library of variant, epitope-specific antibody sequences that have a predicted improved affinity, stability, or the combination thereof over the original epitope-specific antibody sequence, the method comprising the steps of:
a. utilizing computational saturation mutagenesis, identify positions in said original antibody sequence, and mutations at said positions of said original epitope-specific antibody sequence, that satisfy a predefined first threshold of affinity score, stability score of combination thereof compared to that of the original epitope-specific antibody sequence;
b. for each of said positions and mutations at said positions, compute the affinity score, stability score, or the combination thereof, of a plurality of variant antibody sequences having each mutation at each of said positions, and rank the variant antibody sequences having each of said mutations at each of said positions according to a predefined second threshold of affinity score, stability score or combination thereof compared to that of the original antibody sequence; and
c. identify from the affinity score, stability score of combination thereof that is above a predefined third threshold of affinity score, stability score or combination thereof compared to that of the original antibody sequence those variant antibody sequences with a predicted improved affinity score, stability score or combination thereof compared to that of the original antibody sequence.

2. The method of claim 1 wherein in step (b), computation of the affinity score, stability score of combination thereof of the plurality of variant epitope-specific antibody sequences is carried out on multiple computers, such that each computer computes, for a portion of the plurality of variant antibody sequences, the difference in affinity score, stability score, or combination thereof of said subset of variant antibody sequences and that of the original epitope-specific antibody sequence.

3. The method of claim 1 wherein in step (a), said positions that are identified by computational saturation mutagenesis are determined using a position specific scoring matrix (PSSM), wherein one or more replacement amino acids at each position results in a negative, unchanged, or slightly positive affinity score, stability score or combination thereof compared to the amino acid at that position in the original epitope-specific antibody sequence.

4. The method of claim 1 wherein the threshold is based on both the affinity score and the stability score.

5. The method of claim 1 wherein the first threshold of affinity or stability is a predefined value of the difference between the affinity score, stability score or combination thereof before mutation and the score after mutation.

6. The method of claim 5 wherein the first threshold is based on both the affinity score and the stability score.

7. The method of claim 1 wherein the second threshold for affinity or stability is a predefined value of difference in affinity or stability score before mutation and the affinity score or stability score after mutation.

8. The method of claim 7 wherein the second threshold is based on both the affinity score and the stability score.

9. The method of claim 1 wherein the third threshold for affinity or stability is a predefined value of difference in affinity or stability score before mutation and the affinity score or stability score after mutation.

10. The method of claim 9 wherein the third threshold is based on both the affinity score and the stability score.

11. The method of claim 1 wherein the original epitope-specific antibody sequence is obtained by epitope-specific antibody engineering.

12. The method of claim 11 wherein the positions identified in the original epitope-specific antibody sequence are within about 8 A of the antigen.

13. The method of identifying an improved epitope-specific variant antibody sequence comprising the steps of:
a. obtaining a library of variant, epitope-specific antibody sequences that have a predicted improved affinity or stability over the original epitope-specific antibody sequence in accordance with claim 1;
b. generating DNA oligonucleotide sequences comprising the CDR3 sequences of the sequences in said library, suitable for expression in an organism in which binding activity in a display or expression system is to be assessed;
c. expressing the corresponding library of scFVs comprising said CDR3;
d. screening said display or expression system for binding of the epitope and identify therefrom one or more CDR3 comprising an improved epitope-specific variant antibody sequence.

14. The method of claim 11 wherein epitope-specific antibody engineering is carried out by
a. generating one or more seed structures based on one or more predetermined amino acid sequences of a complementarity determining region (CDR), one or more predetermined variable heavy (VH) and variable light (VL) structural framework (VHNL) pairs, or a combination thereof;
b. providing a predetermined epitope;
c. docking said one or more seed structures on said epitope:
d. evaluating one or more motifs of said one or more seed structures for one or more predetermined developability properties; and
e. identifying one or more target structures in order to generate a library, thereby generating a library of antibodies.
